(19) United States
(12) Patent Application Publication
(10) Pub. No.: US 2016/0206848 A1
(43) Pub. Date: Jul. 21, 2016

(54) METHOD OF COMPLEMENTARY ALTERNATIVE MEDICINE FOR MRI ANXIETY

(71) Applicant: Selena I. Glenn, Portland, OR (US)

(72) Inventor: Selena I. Glenn, Portland, OR (US)

(21) Appl. No.: 14/993,279

(22) Filed: Jan. 12, 2016

Related U.S. Application Data
(60) Provisional application No. 62/103,740, filed on Jan. 15, 2015.

(51) Int. Cl. A61M 21/02; A61B 5/055

(52) U.S. Cl. CPC A61M 21/02; A61B 5/0555; A61M 2021/0016

(57) ABSTRACT

A method to reduce patient stress during MRI treatment includes a combination of aromatherapy and breathing techniques. The patient selects select from four different aromatherapy scent blends, all of which include pure undiluted essential oils. The blends include combinations which are fruity, floral, earthy, or minty. The patient is placed on the MRI scanning table and hooked up to breathing bellows and the patient chooses the aromatherapy scent. Then, two drops of the scent are placed on a 2×2 inch gauze and placed in front of an air vent located inside the MRI machine bore. Then, one of any selected breathing techniques begins before and/or during the MRI procedure.

### CROSS-REFERENCE TO RELATED APPLICATIONS

The present application claims the benefit of U.S. Provisional Application No. 62/103,740, filed on Jan. 15, 2015.

### SUMMARY OF THE INVENTION

The present invention relates to a method and treatment protocol for reducing anxiety of human patients undergoing magnetic resonance imaging (MRI) procedures in a clinical setting. More specifically, the present invention relates to breathing techniques and aromatherapy techniques, including the use of essential oils, along with other ordered steps designed to reduce emotional stress when a patient is undergoing various MRI procedures.

In one embodiment of the invention, the method may include providing a patient at least one aromatherapy scent and applying the aromatherapy scent in front of an air vent located on the MRI machine. The method may include placing at least one drop of the aromatherapy scent on a gauze and scent in front of an air vent located on the MRI machine. The method may further include instructing the patient to use at least one breathing exercise. The gauze may be of a 2×2 inch size. The aromatherapy scent may be selected by the patient. The aromatherapy scent may be a blend of two or more aromatherapy scents.

The method may include instructing the patient to engage in a breathing exercise comprising belly breathing and inhaling through the patient's nose progressively. This breathing exercise may also include instructing the patient to extend exhalation through the patient's mouth.

The breathing exercise may include instructing the patient to do a four-count nose inhalation, a seven count breath hold and resistant mouth exhalation. The breathing exercise may include instructing the patient to perform slow, nostril breathing with extended exhalation comprising inhaling for three counts and exhaling for six counts. The breathing exercises may be performed prior to the MRI scan or during the MRI procedure.

In one embodiment, this invention includes an apparatus including an MRI scanning table with an air vent, with gauze attached to the air vent, and at least one aromatherapy scent.

In another embodiment, this invention includes an instructional recording of breathing techniques playable on an MRI sound system.

The foregoing and other objectives, features, and advantages of the invention will be more readily understood upon consideration of the following detailed description of the invention taken in conjunction with the accompanying drawings.

### BRIEF DESCRIPTION OF THE SEVERAL DRAWINGS

FIG. 1: Individual Patient Self-Assessed Pre-SIGBA/MRI, SIGBA, and MRI Anxiety Likert Scales.

FIG. 2: Experimental group with medication whose medication NOT effective before the start of their MRI exam.

FIG. 3: Experimental group with medication, whose medication took effect before the start of their MRI exam.

FIG. 4: Experimental Most Effective Tool

FIG. 5: Qualitative Patient Suggestions

### BACKGROUND OF THE INVENTION

Healthcare costs are ever-increasing. As care-providers attempt to contain and reduce costs, one avenue typically employed includes efficiently scheduling capital-intensive equipment and maximizing patient-treatment bookings on such equipment. One example of capital-intensive equipment includes magnetic resonance imaging (MRI) equipment, which includes a suite of dedicated space and staff to run, maintain, and otherwise operate. However, many patients experience debilitating claustrophobia resulting in canceled appointments for their MRI treatment, resulting in lost revenue and increased overhead costs for the healthcare provider.

As is generally known in the art, complementary and/or alternative medicine (CAM) and cognitive restructuring can reduce the incidence of prematurely cancelled Magnetic Resonance Imaging (MRI) due to anxiety (Lukins, Davan, & Drummond. 1997; Quirk, Letendre, Ciottone, & Lingley, 1989b; Redd, Manne, Peters, Jacobsen, & Schmidt, 1994). However, despite advances in stress reduction in this particular field, there remains yet a need for improved treatment procedures that render more patients more comfortable with MRI treatment, resulting in improved operating efficiencies for the healthcare provider. Further, there is a need for a low-cost, knowledge-based solutions for this issue.

Accordingly, there remains a need for complementary and/or alternative medicine modalities, such as aromatherapy and breathing techniques, to reduce patient anxiety and reestablish emotional and physical homeostasis in patients undergoing MRI examinations. Specifically, there is a need for an effective combination of breathing techniques and aromatherapy to reduce the time it takes medication to take effect in patients whose medication had not reached maximum effectiveness before their MRI appointment.

Further, there is a need for a customer service protocol for training MRI technologists on how to effectively deal with anxious patients using these CAM modalities. This customer service training should incorporate evidence-based solutions for identifying potentially anxious patients and applying CAM and cognitive restructuring techniques to ease their fear. CAM and cognitive restructuring can reduce the incidence of prematurely cancelled Magnetic Resonance Imaging (MRI) due to anxiety (Lukins, Davan, & Drummond, 1997; Quirk, Letendre, Ciottone, & Lingley, 1989b; Redd, Manne, Peters, Jacobsen, & 10 Schmidt, 1994).

Anxiety in the form of claustrophobia during MRI exams is common and continues to be a problem in many imaging departments around the world. It is estimated that nearly 40% of patients that undergo this exam has some form of anxiety ranging from mild apprehension to full blown panic attacks resulting in premature termination of up to 20% of examinations (Goyen & Klewer, 1997; McGlynn, Smitherman, Hammel, & Lazarte, 2007; Mohlman, Eldreth, Price, Chazin, & Glover, 2012). Early exam termination and no-shows from patients who have had previous anxious experiences during their MRI examination interrupts their medical treatment (Sarji, Abdullah, Kumar, Tan, & Narayanan, 1998). Furthermore these cancellations and/or no-shows are costly to the organization due to lost revenue from wasted appointments and labor hours (Murphy & Brunberg, 1997). A 1993 study found an estimated “annual net loss of $65,250,000” (Murphy & Brunberg, 1997, p. 52) from cancelled MRI examinations in the United States of America (Murphy & Brunberg, 1997). A report from the Centers for Disease Control (CDC) stated that from the mid 1990's to 2006 the number of MRI machines in America had nearly doubled (AP, 2010). With this significant increase in the utilization of MRIs, a current estimate of loss revenue from cancelled exams has certainly surpassed the estimate above.

There is room for improvement in patient care in the MRI environment. There is a gap in information available on anxiety in the MRI environment, not only for technologist, but patients as well (Törnqvist, Månsson, Larsson, & Hallström, 2006). The gap of available information on this topic leaves room for innovation and an opportunity to conduct research and approach this phenomenon from an evidenced based perspective. Furthermore, with the ever increasing interest of using CAM by Americans, breathing exercises appeared to be an appropriate path to explore for solutions, because of its low implementation costs, relatively obscure application in medical imaging, and its success in other medical modalities such as nursing and dentistry (Biggs, 2003; Snyder & Lindquist, 2006).

There has been a significant amount of research exploring the incidence of anxiety during MRI exams on a national and global scale. These research topics include exploring the demographics and predicting anxiety sufferers during the exam (Meledez & McCrank, 1993; Murphy & Brunberg, 1997; Sarji et al., 1998). Likewise studies exploring the personal experiences patients have during the exam, literature reviews of interventions and their effectiveness, and the financial impact cancelled exams have on patients and their healthcare have been conducted (Murphy & Brunberg, 1997; Phillips & Deary, 1995a; E. Tornqvist, Mansson, Larsson, & Hallstrom, 2006). While these research projects have made significant contributions in bringing attention to this phenomenon, their focus has been on determining if anxiety was a significant issue, the causes of anxiety, and current interventions. However there have been very few research projects focusing attention on creating new interventions and even less using CAM, which is where my research fills the gap.

Anxiety in the MRI environment is significant and warrants more attention in formal MRI training programs. With significant incidences of cancelled MRI exams, along with its financial impact on healthcare costs additional training of MRI technologists is not only necessary, but imperative to keep healthcare costs associated with this increasingly popular diagnostic imaging modality affordable (Högström & Sverre, 1996; Smith-Bindman, Miglioretti, & Larson, 2008). This research will contribute to widening the information available to technologist and MRI educators on this topic. Moreover the original data from the pilot study will contribute to finding an effective treatment for anxious MRI patients along with helpful tools technologists can use to make the patient experience more positive.

Prescribing medication to anxious MRI patients is a common practice that has risk and thus, innovative alternatives are necessary (Murphy & Brunberg, 1997). CAM provides an alternative to the practice of prescribing narcotics and putting patients under general anesthesia which is expensive and has inherent dangers (Phillips & Deary, 1995). The costs of implementing CAM in imaging departments is significantly lower than many of the current interventions, thus this research project can have a significant social impact on keeping healthcare cost in MRI medical imaging low and available to more people. This research concentrates on cost effective, safe, simple, and clinically effective CAM interventions such as aromatherapy and breathing techniques. These techniques are simple and MRI technologists can easily learn and teach patients with minimal disruption in workflow. Furthermore, because there is not only a need for additional knowledge and interventions on this phenomenon, this research might facilitate the potential for the development of a new form of psychology specific to the MRI environment (Sarji et al., 1998).

The substantial prevalence of anxiety in the MRI environment warrants more information about the potential causes, along with finding additional ways of dealing with it through training opportunities for MRI technologist (Murphy & Brunberg, 1997). Therefore a large gap in training technologists exists beyond the technical component of performing MRI examinations and care for anxious patients. One of the aims of this is to shorten that gap and refocus attention on helping patients, and creating a more holistic patient care approach to this useful imaging modality.

Although formal education for MRI technologists has become popular over the years, most MRI technologist training is still on-the-job training with a large portion of the more seasoned technologists having received this type of education. This training lacks structure and any information passed on concerning anxious patients is purely antidotal and hasn't been thoroughly researched. This invention will contribute to finding research based approaches to MRI anxiety facilitate the spread of evidence based knowledge through the development of continuing educations courses specific to this problem, and making it readily available to on-the-job trained technologists.

In conclusion this invention may make a major contribution to several aspects of treating anxiety in the MRI environment. This includes making more information about helping anxious patients available to technologists in formal training and continuing education. It also contributes to adding an additional intervention for MRI anxiety and an alternative to patients that may not or cannot use sedation. This disclosure adds to the literature of CAM usage and integration into orthodox medicine. It also contributes to CAM usage in the MRI environment, specifically pertaining to using scent and breathing techniques as cognitive interventions, which will be discussed further in a later section.

Anxiety during MRI treatment is well studied and well-appreciated by those having ordinary skill in this art. Some effects of anxiety include the physical and emotional toil on the patient, and the economic impact of this anxiety on the care-giver. Patient anxiety can be understood by understanding the demographics of the patients.

Magnetic Resonance Imaging (MRI) is a diagnostic imaging modality that utilizes a magnet to produce superior images of the human body, and was one of the most important medical imaging developments in the 20th century (Geva, 2006; McConnell & Druva, 2011). Although the discovery of MRI's is usually credited to Paul Lauterbur and Peter Mansfield, there have been a number of other scientists whose work helped lead to the discovery of this technology (Geva, 2006). These scientists include Nikola Tesla, who discovered alternating currents from a magnet, and Jen Baptiste Joseph Fourier a 19th century mathematician who developed the Fourier transform method, which paved the way to create the computer computations necessary for the digital images MRIs produce (Geva, 2006; Westbrook & Roth, 2013). Additionally, Sir Joseph Larmor whose work discovered how the strength of a magnetic field affects the behavior of protons and developed the Larmor equations, which is a basis of MRI technology. Some of the more recent significant contributions include the 1944 Nobel prize of Physics winner, Isidor Rabi a physicist who used Larmor's equation to discover how atoms behaved and who coined the term ‘Nuclear Magnetic Resonance’, (Geva, 2006, p. 574) to describe this technology, which later became known as Magnetic Resonance Imaging (Geva, 2006).

Soon after Lauterbur and Mansfield's discoveries the interest in MRI as a clinical tool in medical imaging amplified (Geva, 2006). In the early 1980's MRI technology began to be used in clinical medical imaging and its popularity soared due to its noninvasive nature, superior soft tissue images, and the lack of dangerous ionizing radiation as used in x-ray imaging. From this point the ground work was laid for utilizing MRIs for a variety of medical imaging needs (Geva, 2006; Westbrook & Roth, 2013).

Not long after the use of MRIs as a diagnostic imaging tool became more common, claustrophobia and anxiety during this exam became prevalent. One of the earliest articles published in 1984, with MRI claustrophobia as the topic, suggested that scanning patients in the prone position would help alleviate their anxiety (Hricak & Amparo, 1984). Another early journal article written by Elizabeth A Klonoff, et al (1986), described a single clinical case study in which a woman with a brain tumor was unable to complete an MRI examination even though it was imperative for a definitive diagnosis and subsequent treatment. As the popularity of MRI imaging increased it became apparent that even though this new imaging modality had great potential, interventions were necessary to ensure patients could endure the examination with relative comfort (Bell, 1996; Hricak & Amparo, 1984; 5 Quirk et al., 1989b)

Prevalence of Anxiety in MRI: Anxiety in the form of claustrophobia and panic attacks during MRI exams are common and continues to be an ongoing problem in many imaging departments around the world. Although MRI examinations are physically non-invasive it has been known to place a considerable amount psychological strain on a significant number of patients in the form of claustrophobia (Chapman, Bernier, & Rusak, 2010; Kilborn & Labbe, 1990). In the early 1990's it was estimated that nearly 40% of patients that undergo this exam has some form of anxiety ranging from mild apprehension to full blown panic attacks requiring premature termination of 2% to upwards of 6.5%, while others reported a failure rate of 10 to 20% (Chapman et al., 2010; Goyen & Klewer, 1997; McGlynn et al., 2007; Mohlman et al., 2012). Out of the 40% of patients who experience MRI anxiety 25 to 37% of them experience moderate to severe anxiety (Chapman et al., 2010).

Although studies estimate the rate of claustrophobia to be nearly 40%, some MRI technologists hold a different opinion. A survey of technologists concentrating on the prevalence of anxiety/claustrophobia in MRI patients estimated up to 71% of their patients experienced anxiety and approximately 19% caused major scanning issues (Chapman et al., 2010; Munn & Jordan, 25 2011; Tischler et al., 2008). One of the ongoing issues is patients often times do not admit or are unaware they are claustrophobic until the exam begins and they start experiencing symptoms of anxiety (Kilborn & Labbe, 1990).

The level of anxiety patients experience varies and is dependent on a number of factors. Some factors includes, their level of knowledge of what to expect, the length of the exam, the size of the machine bore (opening) and whether they fit comfortably, and if the patient has ever had an MRI exam before (Chapman et al., 2010; Mohlman et al., 2012; Thorpe, Salkovskis, 5 & Dittner, 2008; Törnqvist et al., 2006). First time MRI patients appear to have more anxiety then patients who have had previously experience (Chapman et al., 2010). This claim was supported in a study that observed patient's cortisol levels were higher in those who underwent MRI examinations the first time compare to the second scan (Chapman et al., 2010). This study concluded the “habituation” (Chapman et al., 2010, p. 160) of receiving MRI's decreases anxiety, because desensitization occurred (Chapman et al., 2010; Klonoff, Janata, & Kaufman, 1986). Systematic desensitization is a common cognitive therapy approach to treating phobias, which involves exposing the person to the phobic stimulus over a period of time to decrease their anxiety (Craske, Antony, & Barlow, 2006). It is believed that the more examinations a patient has the less likely they will experience anxiety (Chapman et al., 2010; Klonoff et al., 1986).

Inadequate patient education contributes to MRI anxiety. There has been a deficiency of patient education, with false and/or exaggerated ideas about what to expect during an MRI exam (Bolejko, Sarvik, Hagell, & Brinck, 2008; Törnqvist et al., 2006). Formal education for patients about MRI examinations is lacking and the majority of information given has been informal from family member, friends and unfortunately hospital based TV shows that often depicts this exam falsely. The information patients get from friends and relatives is somewhat true, but because one person's experience is unique due to the variability of factors that contribute to anxiety (Mohlman et al., 2012; Phillips & Deary, 1995a). For example, some people may be sensitive to the noise level, while another may find the small space bothersome (McNulty & McNulty, 2009; Mohlman et al., 2012).

To help combat this misinformation, accurate information of what patients can expect during MRI exams is necessary (Bolejko et al., 2008). There is truth in the saying coined by philosopher Sir Frances Bacon “knowledge is power”. Patients who are knowledgeable have a greater sense of control over the situation, as a loss of control is a major anxiety inducer (Bolejko et al., 2008; Thorpe et al., 2008). Furthermore patients have shown interest in receiving more detailed information about the MRI procedures, and supplying this material helps combat false or exaggerated assumptions about the exam and provides an additional tool to conquer their anxieties (Bolejko et al., 2008; Phillips & Deary, 1995).

MRI anxiety is an international problem that affects a large number of people around the world, although it is more significant in North America and Western Europe (Sarji et al., 1998). There are mixed reports of which gender experiences more MRI anxiety. Some studies have shown women are more likely to experience anxiety, while others have reported men to suffer more (Chapman et al., 2010; Sarji et al., 1998; Törnqvist et al., 2006). A person's economic status may play a role in whether or not they will experience anxiety as Sarji et al. (1998) indicated people from higher economic status tends to experience more anxiety.

Age may also play a role into whether a person will experience anxiety during MRIs. Studies have evaluated this issue with varied conclusions. One study concluded that the majority of people who experienced anxiety during MRI examinations were young and between the ages of 25-45 (Sarji et al., 1998). Another found children often times experience high levels resulting in undiagnostic images due to movement, and at times examinations are prematurely cancelled or not performed at all (De Amorim e Silva, Mackenzie, Hallowell, Stewart, & Ditchfield, 2006). For this reason children are often sedated during MRIs (De Amorim e Silva et al., 2006). A different study suggested older adults may suffer from claustrophobia more than younger populations (Murphy & Brunberg, 1997). In a study reviewing the incidences of patients that required sedation found the mean age of patients requiring sedation was 60 years old (Murphy & Brunberg, 1997). A study by Mohlman et al (2012) comparing the difference between patients who suffered from generalized anxiety disorder (GAD) and a control group who were 60 years or older showed the control group had a 29% failure rate of completing their MRI examination, while the GAD group had a 32% failure rate. Conversely a study by Wollman et al. (2004) specifically evaluating MRI anxiety in older adults, and found patients over the age of 90 tolerated the examination well.

In summary, according to the literature it appears that although some patient populations experience more anxiety than others during MRIs, the general consensus exists that anxiety is a common phenomenon amongst patients (Chapman et al., 2010; Mohlman et al., 2012; Sarji et al., 1998).

Cancelled MRI exams have a financial impact on imaging centers, patients and contribute to the increases in healthcare costs (Baker & Wheeler, 1998; Dinan, Curtis, Hammil, & et al, 2010; Högström & Sverre, 1996). The financial impact of cancelled MRI examinations is substantial. For example, it was estimated that in 2011, there was a 2.3% worldwide cancellation rate due to claustrophobia with a “95% confidence interval” (Enders et al., 2011, p. 1), which resulted in nearly 2 million MRI exams being cancelled due to claustrophobia and sustained an estimated a loss of 1 billion Euros which equated to approximately 1.35 billion American dollars (Enders et al., 2011).

The financial impact of cancelled MRI exams has on patients, imaging departments and overall healthcare costs is somewhat complex with many contributing factors (Bell, 1996; Högström & Sverre, 5 1996). Some of these factors include insurance reimbursements, medical legislation and regulations, and lastly operating costs (Bell, 1996; Dinan et al., 2010; Mitchell & LaGalia, 2009). To get a better understanding of the complexities an exploration of the economics of MRI is necessary (Bell, 1996).

The cost of running an MRI machine is dependent on how long the machine has been in service (Baker & Wheeler, 1998; Bell, 1996). The initial cost of buying and installing an MRI machine is more than one million dollars and it takes time to recapture the initial investment (Baker & Wheeler, 1998). The newer the machine the higher costs, with an average cost per exam estimated between $100-$400 (Bell, 1996). This cost is also influenced by where the exam is performed with operational costs of a free standing imaging center being considerably higher than in a hospital (Bell, 1996). The cost per exam is also dependent of how many patients are seen, thus any reduction in the number of daily exams increases the costs (Bell, 1996). Research has concluded that cancelled MRI's decreases the number of exams performed and contributes to increasing the operational costs of imaging centers and ultimately higher healthcare costs (Baker & Wheeler, 1998; Bell, 1996).

The insurance industry has had a significant impact on the economics of MRI departments. These changes have had a financial impact on the operating costs of imaging departments and overall healthcare costs (Mitchell & LaGalia, 2009). The popularity of using advanced imaging such as MRIs increased dramatically in the 1990's, and insurance companies took notice (Mitchell & LaGalia, 2009). Insurance companies then began promoting the current trend aimed at decreasing their costs by reducing the number of advanced imaging tests performed (Mitchell & LaGalia, 2009). This trend has been assisted by Radiology Benefit Management programs or RBMs. RBMs requires preauthorization of any advanced imaging study, by insurance companies before they are performed (Mitchell & LaGalia, 2009). As a result of RBMs there has been a consistent reduction in the number of MRI performed in the areas where these programs are prevalent (Mitchell & LaGalia, 2009).

This trend contributes to the reduction in the number of MRI exams performed and equates to decreasing imaging department's patient volume revenue (Bell, 1996). When there is a reduction in the number of exams performed in a given day due to anxiety, the cost of each exam increases due to costs associated with operating the machine (Baker & Wheeler, 1998). For example if the operational costs of an exam is $100 per exam and there are ten exams scheduled that day, and one MRI is cancelled due to anxiety, the operational costs of the other exams completed goes up to $110 per exam subsequently increasing the overall operational costs (Baker & Wheeler, 1998). This creates a need for imaging departments to become efficient in completing every exam (Bell, 1996).

Substantial reductions in reimbursements insurance companies will pay for MRI exams along with higher insurance premiums have contributed to rising healthcare costs (Bell, 1996; Mitchell & LaGalia, 2009). In the late 1980's the average reimbursement for MRI was approximately $1,000 per exam, the current reimbursement is approximately half of that (Mitchell & LaGalia, 2009). The economic impact of cancelled MRI exams expands beyond medical facilities, and have affected radiologists as well as they have seen a reduction in reimbursements for their professional services (Bell, 1996). This reduction in both the volume of exams radiologist read, and insurance reimbursements may cause a domino effect. Radiologists may charge more for their professional services to make up for the difference in revenue, which contributes to an overall increase in health care costs (Bell, 1996). Consequently an increase of performing advanced imaging techniques such as MRIs was cited as a contributing factor to higher insurance premiums (Bell, 1996; Mitchell & LaGalia, 2009). Thus one of the ways of reducing healthcare costs, imaging facility overhead, and insurance premiums is to ensure efficiency in MRI departments.

The trend of trying to reduce the utilization of advanced imaging extends beyond private insurers and into government programs. Over the last three decades increasing government regulations regarding the use of MRIs have played a role in increasing operational costs of imaging centers (Bell, 1996; Smith-Bindman et al., 2008). The federal government has joined private insurance companies and has reduced Medicare and Medicaid reimbursements via the Deficit Reduction Act of 2005 (Smith-Bindman et al., 2008). The Deficit Reduction Act took place in 2005 drastically reducing reimbursement of MRI exams (Smith-Bindman et al., 2008). For example the rate of reimbursement for a brain MRI for a free standing imaging center was $995. After enactment, reimbursements were reduced to $506 for the same exam (Smith-Bindman et al., 2008). That was approximately a 50% reduction in reimbursements, which in turn increased the cost per exam, because costs associated with performing an MRI remained the same (Mitchell & LaGalia, 2009; Smith-Bindman et al., 2008).

Another government regulation that decreased the utilization of advance imaging was the Certificate-of-Need, which limits the number of MRI units in a particular area and is based on the community's needs. While the Certificate of Need regulation helped reduce the number of excessive MRI units in communities, it also inadvertently drove up the cost per exam of advanced imaging and created a need for efficiency (Högström & Sverre, 1996; Mitchell & LaGalia, 2009; Smith-Bindman et al., 2008).

The financial impact of cancelled MRI exams also extends to patients. The financial impact on patients when they cannot complete their exam has not been a topic thoroughly discussed in the literature. Although the financial impact on patients is not as high as imaging departments, there is still an impact. The most obvious financial cost to patients is transportation to their MRI appointment. When their exam is prematurely cancelled, transportation costs essentially double (Högström & Sverre, 1996). Transportation costs can also be considered a contributing factor in the increasing healthcare costs, as the elderly, low income and disable patients sometimes depend on government funded transportation in the form of vouchers, and repeated trips results in not only wasted gas, but wasted man hours of the drivers repeating the same route.

Moreover patients may take time off from work in order to get their MRI exam and may sustain financial losses, as it is estimated that less than half of all workers receive paid sick time (Lovell, 2004). When they cannot complete their exam, the time they took off is essentially wasted, and additional income loss may incur as they eventually have to return for another appointment that may or may not be covered by their medical sick hours. Lastly cancelled MRI exams can sustain a financial loss due to delayed medical care due to the progression of an injury or illness, which in turn increases medical expenses and additional time from work (Högström & Sverre, 1996; Lovell, 2004).

With the continuously decreasing reimbursements from insurance companies, government funded medical programs, an increased regulations placed on performing advance imaging, and the changes in American healthcare policies trending towards socialized medicine there is a need for more efficiency in MRI imaging (Bell, 1996). Furthermore early exam termination and no-shows MRI examinations has financial impacts on patients, and more importantly it interrupts their medical treatment (Högström & Sverre, 1996; Sarji et al., 1998).

The theoretical foundations this study is divided into two categories, in an effort of fully encompass the complex nature of anxiety in the MRI environment and provide a multidimensional approach to this issue. These theoretical foundations were chosen after conducting a literature review on the theoretical schools of thought on integration of CAM into orthodox medicine and psychological counseling for anxiety disorders. The first discussion involves the integrative medicine theory, based is the belief that when treating patients with an integrative medicine approach creates a more holistic approach to medicine (Boon, Verhoef, O'Hara, & Findlay, 2004). The reasoning for choosing this approach was, not every treatment approach will be successful for every patient, creating the need for a multidimensional approach.

The second theoretical basis of this study was the integration of humanistic and cognitive behavior theories. The reasoning for choosing this theoretical approach was that successful completion of an MRI examination is a team effort between the technologist and patient alike, and their interpersonal relationship can affect the exams completion (Tornqvist et al., 2006). Integration of humanistic and cognitive theories consists of combining two theories, for this reason each theory will be explored separately along with how it applies to this study and MRI anxiety, followed by their integration and how it applies to the MRI environment will be discussed.

The theoretical school of thought as applied to this study leading to the present invention is the integrative theory which was used to support introducing CAM into the MRI environment. The integrative theory described as a “non-hierarchical” (Boon et al., 2010, p. 3) blending of CAM, and orthodox medicine, thus treating the patient as a whole to include their mind, body and spirit, and creating mutual respect between CAM and orthodox medicine (Boon et al., 2010). The integration of an allopathic diagnostic tool (MRI) and CAM approaches in treating MRI anxiety creates a “conceptual framework” (Miller, Sward, Nielsen, & Robertson, 2011, p. 5) when the theoretical approaches of these two approaches to medicine are integrated a more effective outcome is produced then either one alone (Miller, Sward, Nielsen, & Robertson, 2011). That being said, there have been many different approaches to treating anxiety in the MRI environment and integrating all of these interventions to include both CAM and non-CAM approaches will be more effective to the patient than using one. Additionally there has been a trend toward more holistic medicine encompassing a variety of techniques to treat patients making the applications of this theoretical approach to treating anxious MRI patients in line with a currently accepted approach to medicine (Carrol, 2010; Keshet, Ben-Arye, & Schiff, 2012). Lastly this approach to treating MRI anxiety goes beyond the one size fits all approach and takes the differences in how people respond differently to treatments and varying reasons anxiety during the exam (De Amorim e Silva et al., 2006).

There has been a gradual integration between CAM and conventional orthodox medicine (COM) over the years due to the general public's interest in finding alternative and/or complement to COM (Carrol, 2010). This is evident in the fact that over 40% of Americans have used some form of CAM in their health care with millions of out of pocket dollars spent on CAM therapies spent every year by patients. (Carroll, et al., 2010). One reason was the introduction of Eastern medicine systems to the West (Cant, 1998; Gonzales et al., 2010; Stringer & Donald, 2011; Thompson & Coppens, 1994). Another is patients have been moving towards more natural medicine and have interests in returning to the medical practices of their ancestors (Carroll, et al., 2010).

While CAM has been introduced to areas in conventional orthodox medicine such as in nursing, surgery and palliative care, there are still many areas in which CAM can make a difference and fulfill the public's demand for healthcare alternatives that benefit patients (L. J., 2006; Stringer & Donald, 2011; Tusek, Church, & Fazio, 1997). Including MRI imaging amongst those medical modalities encompasses a more holistic approach is important as it serves as one of the most popular noninvasive, diagnostic tools amongst medical practitioners (Chapman et al., 2010). CAM modalities such as music therapy, guided imagery and aromatherapy have been used in the medical specialties mentioned above and there is no reason it cannot be extending into patient care in MRI (Chan, Lee, Ng, Ngan, & Wong, 2003; Stringer & Donald, 2011; Tusek, Church, & Fazio, 1997).

Integrating CAM into medical imaging helps increase awareness of the different CAM modalities and creates educational opportunities for both COM and CAM practitioners. Integration of these medical philosophies is important because both contributes and is committed to providing good patient care, therefore setting the stage for them to coexist creates more holistic approach to medical care. Their integration can improve patient care, which is a key factor in creating a bridge between these two medical practices (Cant, 1998).

The humanistic theoretical approach focuses on the interpersonal relationship between the patient and counselor, emphasizing a positive, safe and trust oriented relationship (Miller, Sward, Nielsen, & Robertson, 2011b). Trust is the basis of any successful relationship and the relationship between the MRI technologist and patient is no different. A quantitative study examining patient's perceptions of receiving an MRI, concluded that the relationship between that patient and technologist greatly influenced the patients overall experience (Tornqvist, Mansson, Larsson, & Hallstrom, 2006). Thus there is a need for patients to trust the MRI technologist will keep them safe and out of harm's way. This is an important factor because when a patient is in the MRI environment they may begin to feel uncomfortable, and/or unsafe as the MRI environment can be intimidating (Enders et al., 2011; Munn & Jordan, 2011). These factors include the MRI machine's appearance, as the machine is very large relative to the machines bore, which is the area patient lays, and is relatively small in comparison (Munn & Jordan, 2011). When the machine is acquiring images it produces a very loud sound similar to a jack hammer and sound vibrations can be felt, which some patients may find disturbing (Lukins, Davan, & Drummond, 1997; McNulty & McNulty, 2009; Thorpe et al., 2008). Sometimes patients experience nerve stimulation, and at times involuntary muscle contractions (Westbrook & Roth, 2013). These unusual and involuntary physical sensations contribute to patients feeling they are not being in control and adds to the overwhelming feelings (Tornqvist et al., 2006). Patients have also had feelings of loneliness and felt unsupported in the exam room creating vulnerability and which a trusting relationship of the humanistic approach can help patients feel more supported and less defenseless (Robyn Lukins et al., 1997; Thorpe et al., 2008; Tornqvist et al., 2006).

When patients begin to experience anxiety the technologist often takes on the role of consoling the patient and in essence taking on the role of a psychological counselor to keep them calm. This is where the humanistic theory comes into play as one of the primary responsibilities of the technologist is to make the patient feel well taken care of. In order for that to happen, a positive interpersonal relationship between then is necessary, which the humanistic approach provides (Miller, Sward, Nielsen, & Robertson, 2011a). Studies found an increase in treatment adherence by patients when there is good rapport between the technologist and other medical personal (Lang, Ward, & Laser, 2010; Thorpe, Salkovskis, & Dittner, 2008). Thus focusing on the patient's needs may enable them to make the healthy choice of completing their exam (Miller et al., 2011).

The cognitive therapy approach can be used in the MRI environment to help patients control negative cognitions when experiencing anxiety (Thorpe et al., 2008). The cognitive therapy approach focuses on educational aspects of psychological treatments to change a person's irrational catastrophic cognitions or thoughts that caused them anxiety (Miller et al., 2011; B. A. Root, 2000). This approach can be applied to an anxious MRI patient's irrational cognitions and aide in reducing exam cancellations due to anxiety, as negative thoughts play a major role in MRI anxiety (Saji et al., 1998; Thorpe et al., 2008). Moreover a correlation between the numbers of negative cognitions was found to be an important factor of whether a patient had anxiety during their MRI (Thorpe et al., 2008). The more common irrational ruminating cognitions anxious MRI patients have include the thoughts; ‘I will be trapped’ ‘I will suffocate’ and ‘I will lose control’ (Thorpe et al., 2008, p. 1082) increasing the likelihood of their inability to complete their exam (Thorpe et al., 2008). This is where the educational aspect of the cognitive theory comes into play. With the technologist's help, patients can be trained to help reduce these negative thoughts. Additionally the technologist becoming educated on the different cognitive approaches that may be helpful in the MRI environment. For example, to help patients with negative cognitions MRI technologist can coach patient to relax such as suggesting positive thoughts and encouraging them to use breathing techniques during the breaks in imaging sequences (Lukins et al., 1997). Additionally patients can be educated on the role cognitions play in their anxiety giving them a tool to help self-regulate their emotions.

There is a definitive correlation between a patient's claustrophobia and cognitions. Thus, incorporating the cognition theory into this study is logical. (Thorpe et al., 2008). The idea of cognitions playing a role in MRI anxiety along with using the cognitive approach to help MRI patients has been explored in several studies. Several studies have tested this approach with positive results, the most recent study conducted in 2008 and another in 1997 along with an individual case study (Lukins et al., 1997; Thorpe et al., 2008). Their approaches to cognition's role in MRI were different. The former study focused on how cognitions contributed to anxiety and the two later studies on how cognitions could be used as an intervention. The study focusing on using cognition training as an intervention incorporated relaxation techniques and concluded cognitive training for anxious patients was helpful in reducing their anxiety (Lukins et al., 1997). During this study a variety of cognitive techniques were employed including muscle relaxation, autogenic suggestions, encouraging patients to use positive affirmations, guided imagery and incorporated the sounds of the MRI sequences (Lukins et al., 1997). An interesting fact revealed in this study was cognitions had the greatest effect on patients who had the most anxiety (Lukins et al., 1997). This finding is significant as patients with the most anxiety are more likely to prematurely end their examination (Thorpe et al., 2008).

The study of cognitions and how they contribute to MRI anxiety has seen resurgence since the initial study conducted in the late 90's. This may be due to the increasing popularity of holistic medicine. The study by Thorpe et al. (2008) exploring the role cognitions have on claustrophobia and panic attacks in the MRI environment concluded cognitions plays a role in patient's psychological disturbances and may be used to create effective interventions for anxious patients. In essence if a claustrophobic person can gain control over their cognitions, their bodies will respond decreasing the uncomfortable physical sensations associated with claustrophobia and panic attacks (Thorpe et al., 2008). Lastly, brief cognitive treatments has been shown to be is just as effective as extensive treatments, thus being able to apply them on the spot with anxious patients makes this a better interventions when comparing it to other psychological treatments for phobias such as progress desensitization (Thorpe et al., 2008).

The integration of the humanistic and cognitive behaviors theories can have a positive influence on the relationship between the MRI technologist and patient. Both theories relate to MRI anxiety, as a successful exam is dependent on the interpersonal relationship between the technologist and the patient's cognitions (Thorpe et al., 2008). The integration of these theories takes the two most important aspects of completing an MRI examination into account, the patient's cognitions and the relationship between the patient and MRI technologist. Additionally it affords a more holistic approach of dealing with anxiety in the MRI environment (Boon et al., 2010; Miller et al., 2011b). In summary, MRI was one of the most significant medical contributions in the 20th century that has enables more accurate diagnosis of a range of illness, but like any major medical development there are possible impediments that may need to be addressed (Chapman et al., 2010; Geva, 2006b; Kilborn & Labbe, 1990). MRI's impediment is the prevalence of anxiety in the form of claustrophobia and anxiety attacks, which can have a significant impact on both patients and imaging centers alike. Although the impact anxiety has on patients goes beyond finances and extends into the quality of healthcare they receive. While anxiety affects some more than others the effects of cancelled exams are the same, a reduction in the quality of care. Thus there is a need for different approaches to helping patients complete their MRI examination and receive the best quality care available to them.

There has been extensive research found on anxiety in the MRI environment. This literature review includes a general discussion of anxiety physiology, the common anxiety disorders found in the MRI environment along with of the specific physiology associated with these anxiety conditions. A discussion of the interventions used to help combat MRI anxiety will follow. Lastly, an in depth discussion of the literature about the correlations between aromatherapy, breathing exercises and cognitive restructuring is included.

The physiology associated with anxiety primarily takes place in the area of the brain that controls our emotions, called the limbic system (Root, 2000). The limbic system controls our emotions through biochemical reactions regulated by a group of structures that intercommunicates with one another (Brown & Gerbarg, 2005a; Root, 2000; Starcevic, 2004). These structures include the prefrontal cortex, hippocampus, thalamus, periaquital grey matter, pineal gland, the olfactory region and amygdala (Root, 2000; Starcevic, 2004). Three structures in the limbic system, the prefrontal cortex, olfactory region and amygdala play a significant role in this thesis due to their regulatory effect on anxiety (Root, 2000; Starcevic, 2004).

The amygdala and its internal structures' role in emotion regulation are significant as they control two major branches of the autonomic nervous system, which are the parasympathetic and sympathetic nervous system (Root, 10 2000; Starcevic, 2004). These branches of the nervous system primarily govern the involuntary bodily functions such as blood pressure and heart rate (Blessing & Gibbins, 2008). A structure located within the amygdala, called the central nucleus controls emotions by regulating the heart and breathing rates, and blood pressure, and are major players in mood regulation (Root, 2000; Starcevic, 2004). Furthermore the central nucleus is the area of the brain anti-anxiety medications target to reduce panic attack symptoms, making it an significant player in anxiety regulation (Root, 2000).

The amygdala may contribute to MRI anxiety as it is responsible for receiving and interrupting visual, and auditory stimulus and determines if an environment is safe or unsafe (Filley, 2011). The amygdala's role in MRI anxiety is clear as it is one of the main limbic structures associated with anxiety disorders and responsible for interpreting threatening sounds (Filley, 2011). This correlation is supported by a study that determined the loud noise of MRI machines as a contributing factor in patient stress (Enders et al., 2011; Filley, 2011; McNulty & McNulty, 2009). The amygdala has direct connections between other parts of the limbic system that regulates emotions, such as the hippocampus, thalamus, periaquital grey matter, and prefrontal cortex (Starcevic, 2004).

The prefrontal cortex is regulated by a branch of the autonomic nervous system and regulates the brains reactions to dangerous situations (Brantley, 2007). The autonomic nervous system consists of two major branches, the sympathetic nervous system (SNS) and parasympathetic nervous system (PNS) (Blessing & Gibbins, 2008; Brantley, 2007). The PNS is responsible for activating the body's relaxation response, while the SNS prepares the body for action when under a perceived or real threat, often referred to as the fight-or-flight reaction (Blessing & Gibbins, 2008; Brantley, 2007). The prefrontal cortex is responsible for turning off the fight-or-flight mechanism of the sympathetic nervous system, which is important when a patient perceives an MRI as a threat (Brantley, 2007; Brown & Gerbarg, 2012).

The olfactory region of the limbic system plays a major role in anxiety regulation. Although the olfactory portion of the limbic system plays an important role in this thesis due to aromatherapy being a major topic, this will be discussed in greater detail in the aromatherapy section of this chapter.

The limbic system produces and regulates the biochemical reactions that control emotions (Blessing & Gibbins, 2008). Neurotransmitters are a result of these biochemical reactions that governs emotions such as anxiety (Root, 2000; Starcevic, 2004). Neurotransmitters essentially act as communicators by sending messages throughout the body via neuron synapses (Root, 2000). The neurotransmitters serotonin and gama aminobutyric acid, (GABA) are produced by the pineal gland and have inhibitory effects on anxiety. This inhibitory effect triggers a part of the PNS responsible for the relaxation response in the body, and in turn calms the fight or flight response when danger is perceived (Root, 2000; Starcevic, 2004). The neurotransmitters serotonin and GABA play an important role in emotions as low levels of either contributes to anxious feelings (Root, 2000). Norepinephrine is another neurotransmitter involved in emotions and is produced by the adrenal gland (Root, 2000). Norepinephrine has the opposite effect of serotonin and GABA, as it keeps the body alert and ready to take action when under threat, thus when it is overproduced it can cause anxiety (Root, 2000).

The two primary disorders experienced by MRI patients are claustrophobia and panic attacks, while agoraphobia is secondarily seen, as it is sometimes coupled with panic attacks (Grey, Price, & Mathews, 2000; Sarji et al., 1998; Starcevic, 2004).

Claustrophobia: Claustrophobia defined as an extreme fear of small enclosed spaces is believed to be the primary reason MRI patients has anxiety and unable to complete their exam (Grey et al., 2000; Sarji et al., 1998; Starcevic, 2004). A person who suffers from claustrophobia fears they will become trapped in an enclosed space and are accompanied by catastrophic thoughts or cognitions, which cause them to panic (Starcevic, 2004). Once a person begins to panic they begin to experience physiological symptoms such a shortness of breath, which is the most prominent symptom associated with claustrophobia (Craske et al., 2006; Starcevic, 2004). When a person experiences respiratory distress they begin to have cognitions that they will “suffocate and die” (Starcevic, 2004, p. 192), which is what happens when patients are in the MRI unit (Craske, Antony, & Barlow, 2006). This fear of suffocating may cause the patient to feel they are suffering from “air hunger” (Starcevic, 2004, p. 198), which is when the body gives a false alarm that it isn't receiving enough oxygen (Starcevic, 2004). The person then begins to breathe faster, and can lead to hyperventilation and ultimately a panic attack (Root, 2000; Starcevic, 2004).

Panic Attacks: A panic attack is characterized as an intense anxiety “episode” (Root, 2000, p. 2) that seemingly comes on without warning. Panic attacks reach climax in a matter of minutes, and generally last between (10) ten to (20) twenty minutes, but can last for an extended period of time (Root, 2000). Although the duration of panic attacks are relatively short, symptoms are perceived as lasting considerably longer to the person experiencing them (Root, 2000). Panic attacks are often experienced by people suffering from a variety of psychiatric disorders including phobias, and in some cases develop phobias due to having a panic attack (Root, 2000; Starcevic, 2004). There are three types of panic disorders including; 1. unexpected, which are those that happen without warning, 2. situational bound that always manifest in a specific situation, and 3. situationally predisposed which is when the likelihood of experiencing an anxiety episode increases in certain situations (Root, 2000; Starcevic, 2004). As a technologist, my observations are patients suffer from all three types of panic attacks in the MRI environment. Some patients know they have anxiety in the machine from prior experience, while some are completely taken by surprise and others only experience panic feelings during MRI exams.

The symptoms associated with a panic attack are considered either primary or secondary signs and are categorized by the frequency in which they occur (Starcevic, 2004). Primary symptoms include, a racing heart and/or heart palpitations, shortness of breath, a choking feeling, uncontrollable shaking, sweating and dizziness, while secondary symptoms include nausea, hot and/or cold flashes, and finally tingling or numbness in extremities (Root, 2000; Starcevic, 2004). These physiological symptoms are governed by the autonomic nervous system, specifically the central nucleus of the amygdala, and the patient is unable to control them. Although patients have no control over the physiological symptoms some have successfully used slow breathing to influence reducing them (Starcevic, 2004). The physiological symptoms of panic are important, as a study showed the majority of MRI patients were more disturbed by the physical symptoms associated with the panic attacks then the MRI exam itself (Thorpe et al., 2008). Furthermore, similar to claustrophobia, the onset of panic attack symptoms often causes catastrophizing thoughts, which exacerbates anxiety (Root, 2000; Starcevic, 2004).

When a patient has a panic attack there is an overwhelming urge to react and leave the situation, thus patients knowing they can escape the situation can mean the difference between them feeling safe or unsafe in the MRI environment (Root, 2000; Starcevic, 2004). Creating a safe environment is often accomplished in MRI by giving patients a panic button, which gives them the “perception” (Reinecke, 2010, p. 25) they have control over the situation, thus reduce their worrisome cognitions (Reinecke, 2010).

Agoraphobia: Agoraphobia is a psychiatric disorder that is closely associated with panic attacks, and sometimes considered a complication of this anxiety disorder, which was the reasoning for its inclusion in this section (Root, 2000). Moreover, based on the characteristics of the disorder it may contribute to no-shows for MRI appointments. Instead of having the urge to flee a perceived dangerous situation agoraphobics tend to avoid the situation all together (Starcevic, 2004). A characteristic of agoraphobia sufferers is their cognitions tend to gravitate towards worrisome thoughts of future events resulting in them going out of their way to evade potentially anxiety causing situations (Root, 2000). Prominent worries for an agoraphobic includes feeling they may be entering a situation they have no control over and that medical help may not be available if they experience a panic attack (Starcevic, 2004). This is an important factor as the majority of patients who experience anxiety during MRIs feel a loss of control and isolated (Thorpe et al., 2008; Tornqvist et al., 2006).

In conclusion, claustrophobia and panic attacks are major contributors to MRI anxiety, and to a lesser extent agoraphobia that needs to be addressed (Harris, Robinson, & Menzies, 1999; Thorpe et al., 2008; Tischler, Calton, Williams, & Cheetham, 2008). While claustrophobia and panic attacks with or without agoraphobia are separate anxiety disorders their effect on MRI patients are the same in contributing to cancelled MRI exams (Thorpe et al., 2008).

Since the growing popularity of MRIs there has been a shift away from the technical aspects of this imaging modality and a focus on more patient centered approaches of addressing anxiety during this examination (Munn & Jordan, 2011). Throughout the years there have been a number of interventions employed to help ease anxiety in MRI patients. These interventions have ranged from extremely high tech interventions such as using virtual reality and goggles to view movies, to low tech solutions, such as changing patient positioning during imaging (Botella et al., 1998; Hricak & Amparo, 1984; Poling, 2014). There have also been time consuming interventions such as systematic desensitization to more immediate mediations such as supplying emergency buttons to patients (Klonoff et al., 1986; Munn & Jordan, 2011). Regardless of the varying types of interventions anxiety in the MRI environment is prevalent and this section will cover a broad range currently in practice (Tischler et al., 2008).

One of the earliest published interventions found during this literature review was a study from 1984 which suggested positioning patients in the prone position (on their stomach) with their eyes facing out of the MRI bore as a simple solution to anxiety (Hricak & Amparo, 1984). This study also suggested covering the patient's eyes in lieu of using such “complicated devices” (Hricak & Amparo, 1984, p. 1) as music from air compressed headphones (Hricak & Amparo, 1984). This intervention was tested, and all patients who used this intervention were able to complete the exam without claustrophobic symptoms (Hricak & Amparo, 1984). While this intervention proved effective, not every patient can be positioned in this manner. For example patients receiving head MRIs cannot be positioned face down. This position may actually exacerbate anxiety as it restricts breathing with is important in controlling anxiety (Brown & Gerbarg, 2012). Furthermore some patients may not able to comfortably hold this position due to physical restrictions such as having a large body habitus, injury or illness. In short this intervention while effective only helps a limited number of anxious patients. That being said, this study did demonstrate that enabling patients to see outside of the bore was useful in reducing MRI anxiety and subsequently contributed to the creation of another intervention with the same concept (Hricak & Amparo, 1984).

This idea was taken one step further in introducing the use of prism mirrors enable seeing outside of the MRI bore and are commonly used in most head coils patients (Munn & Jordan, 2011; Phillips & Deary, 1995). Prism glasses are also available for use with other types of MRI exams (Munn & Jordan, 2013).

The idea of giving patients something else to look at was the premise of a case study investigating virtual reality as an intervention. This study included two known claustrophobic participants and tested the effectiveness of using virtual reality verses music in reducing anxiety in a mock MRI examination (Garcia-Palacios, Hoffman, Richards, Seibel, & Sharar, 2007). The patient with virtual reality as an intervention was able to complete the ten (10) minute mock exam, while the other with music was not (Garcia-Palacios et al., 2007). Although virtual reality helped the claustrophobic patient complete the examination this study only involved two participants thus there is no significant statically power in the study and the results antidotal. Moreover this study was conducted in a simulated MRI machine, which is similar to a real MRI exam, but not equal. For example a real MRI examination usually lasts considerably longer than ten (10) minutes, and patient anxiety due to a possibly negative diagnostic outcome does not exist in a mock situation (Lukins et al., 1997)

Patient education in the form of written information given to patients before their exam has been used to as an intervention to help reduce anxiety in MRI patients. The use of written information was shown to help patients prepare mentally for medical procedures and supports feelings of being in control (Bolejko et al., 2008). Some studies have shown written information helped reduce MRI patient anxiety, motion artifacts and cancelled MRI examinations due to anxiety (Bolejko et al., 2008). Two studies using written information as an intervention were evaluated. The first was a quantitative study conducted in 2004 by Törnqvist, et al. (2006) and evaluated whether written information would reduce MRI patient anxiety and motion artifacts. Both control and experimental groups were given written information with the experimental group receiving more than the control group (Törnqvist et al., 2006). The results showed neither group in the study had any significant difference in anxiety, however the intervention group had less motion with only 4% versus the control group with 15% (Törnqvist et al., 2006).

The second study published in 2008 took a slightly different approach with a qualitative design and provided patients with an informational booklet before their MRI examination (Bolejko et al., 2008). The results showed patients expressed the need more information about MRI procedures and appreciated being given the booklets (Bolejko et al., 2008). Although the main purpose of this study was to evaluate how patients felt about being provided written information, some study participants felt the booklet helped reduce their anxiety (Bolejko et al., 2008).

Both studies had limitations, as neither included written information with coping skills to help manage their anxiety during the exam which have been shown to be an effective intervention (Grey et al., 2000; Munn & Jordan, 2011). For example another MRI anxiety study used a combination of written information along with providing patients with psychological coping skills, which showed positive results in reducing exam apprehension (Grey et al., 2000). Furthermore the Bolejik (2008) study had a small sample size and there did not appear to include a control group. In summary, the current research on written information showed it prepared patients for their exam and may help reduce MRI anxiety, but more research in necessary as supplementing or combining it with other interventions may make it more effective (Bolejko et al., 2008; Grey et al., 2000).

Sedation is currently one of the most popular interventions for MRI anxiety (Phillips & Deary, 1995). Both oral and intravenous sedation use is common practice, and general anesthesia to a lesser extent for adults, but often used for children (De Amorim, Mackenzie, Hallowell, Stewart, 5 & Ditchfield, 2006; Tornqvist et al., 2006). Although sedation is commonly used to help patients tolerate an MRI examination there are inherent risks involved with its use. These risks include drug allergies, being prescribed an ineffective dosage and potential respiratory distress (Bluemke & Breiter, 2000; Phillips & Deary, 1995; Tornqvist 10 et al., 2006). The time it takes a medication to reach maximum effectiveness varies greatly from patient to patient, thus there are occasions when patients must undergo their exam even though the medication has not taken effect (Phillips & Deary, 1995). Moreover some patient populations are unwilling to take medications due to personal preferences or unable to due to prior substance abuse issues risking their sobriety. Others may have adverse reactions to such drugs not have proper post exam transportation making an alternative necessary (Phillips & Deary, 1995).

There were a number of studies looking at sedation in the MRI environment. Although the majority of studies focused on using sedatives for children two were found with adults as the main focus. The first study investigated the incidences of sedation used to help patients tolerated MRI examinations in a hospital setting (Murphy & Brunberg, 1997). It was determined that just over 14% of their patients required sedation with possibly more, as it could not be determined how many patients were prescribed medication by their doctors prior to their examination (Murphy & Brunberg, 1997). A second study focused on the effectiveness and safety of conscious sedation for MRI patients (Bluemke & Breiter, 2000). This study noted a high exam completion rate of 93% in patients who received sedation. Although a small number of complications from the sedation where reported, it was concluded that conscious sedation was safe when administered by a nurse (Bluemke & Breiter, 2000). Statistically speaking this study was powerful as it was conducted over an eight (8) year period with over 4,000 participants (Bluemke & Breiter, 2000). The only limitation found in this study was not having a control group; otherwise this was a strong study.

These two studies gave a good picture of the prevalence and effectiveness of sedation for anxiety in adults undergoing MRI examinations. As stated by the Murphy and Brunberg (1997) study, in addition to using sedation finding other ways of dealing with MRI anxiety is necessary. With this in mind and on the basis of theoretical integration of conventional orthodox medicine and CAM, my study's aim was to fill this gap by focusing on using CAM as either an alternative to sedation and/or complement to oral sedation.

Several environmental interventions have been helped reduce MRI anxiety. This includes creating a more comfortable environment inside the MRI magnet, such as making sure there is plenty of light and good air flow (Munn & Jordan, 2011; Phillips & Deary, 1995). Another way of helping patients feel more comfortable was giving them a sense of control, by providing call buttons making communication between them and the technologist easier (Munn & Jordan, 2011; Phillips & Deary, 1995).

Other interventions have included changes to the MRI machine itself to help patients feel more comfortable. Changes in the machine's design included creating open bore design, instead of the traditional tunnel design (Spouse & Gedroyc, 2000). A literature review looking at the efficiency of this type of magnet design showed it was effective in reducing anxiety in patients (Munn & Jordan, 2011). However all of these studies were conducted on specific groups and not the average MRI patient making it difficult to know how the average patient's anxiety would be affected (Munn & Jordan, 2011). Other limitations to the open design were noted in another literature review that stated the image quality not being as good as that of the traditionally (tunnel) designed magnets (Munn & Jordan, 2011: Phillips & Deary, 1995). Additionally the overall scan time for each examination was increased which poses its own problems, as long scan times is a contributing factor to anxiety (Phillips & Deary, 1995).

Another magnet design used to reduce patient anxiety was the creation of a shorter and wider MRI scanner (Munn & Jordan, 2011). This type of design was used for newer scanners and was shown to reduce both anxiety and the need for sedation (Hunt et al., 2011; Munn & Jordan, 2011). A study conducted over seven (7) months evaluating whether patients who had previously canceled MRI exams performed in a 60 cm bore could tolerate the procedure without general anesthesia in a 70 cm bore (Hunt et al., 2011). The results showed 89% of the patients scheduled in the 70 cm bore successfully completed their exam without anesthesia while only 11% had to use sedation (Hunt et al., 2011). This study concluded that the use of a wider bore machine was beneficial in reducing the use of general anesthesia and increased the likelihood of anxious patients successfully completing their MRI examination (Hunt et al., 2011).

There have been a number of non-CAM interventions used to help anxious MRI patients complete their examination. Some interventions had positive results while others did not and still others were shown to be marginally effective.

Complementary alternative medicine has become increasingly popular in American culture and around the world (Chu & Wallis, 2007; Kessler et al., 2001; Lee, Wu, Tsang, Leung, & Cheung, 2011). An estimated 70% of Australians use CAM, in the UK just over 46% of the population have used it, while between 51% to 82% Taiwanese uses CAM (Chu & Wallis, 2007; Cooke, Mitchell, Tiralongo, & Murfield, 2012). In the USA approximately 40% of Americans have used some form of CAM in their health care with millions of out-of-pocket dollars spent on these therapies (Carrol, 2010). The popularity and financial lucrativeness of CAM has not gone unnoticed by conventional orthodox medicine practitioners, who have begun incorporating it into their practices (Astin, Marie, Pelletier, Hansen, & Haskell, 1998). A literature review of doctors showed there is a considerable number who either practiced CAM techniques with their patients or referred them to CAM practitioners (Astin et al., 1998). Nurses have also begun incorporating CAM into their practices, as a survey of critical care nurses concluded its use was commonly seen as a positive for their patients, with 93% who said it was helpful in easing anxiety (Cooke et al., 2012). Complementary medicine has been shown to be an effective and a popular way of treating anxiety (Kessler et al., 2001). A survey study by Kessler et al (2001) showed CAM to be a common way of treating anxiety amongst Americans. The study was conducted from 1997-1998 with a total of 2,055 participant assessing the incidence of CAM use by adult Americans and found 56% of those surveyed used CAM for anxiety (Kessler et al., 2001). This survey also showed those who used CAM for anxiety declared the therapies were just as effective in treating anxiety than conventional medicine (Kessler et al., 2001). Another study conducted by the Centers for Disease Control (CDS) in 2002 found that over 62% of participants used CAM, with anxiety being amongst the most popular ailment treated (Barnes, Powell-Griner, McFann, & Nahin, 2004). The growing acceptance of CAM and its gradual integration into conventional orthodox medicine (COM) along with research supporting its effectiveness in reducing anxiety reinforces its usefulness in the MRI environment as an intervention (Carrol, 2010; Kessler et al., 2001; Wolpe, 2002).

CAM modalities have been used as interventions in the MRI environment, yet the literature has been somewhat limited. The current literature and/or research on CAM based interventions in the MRI environment include music, aromatherapy, breathing techniques and a variety of other 5 cognitive based therapies such as guided imagery techniques amongst others.

The majority of the literature on CAM use in the MRI environment focuses on the use of cognitive based interventions and their role in patient anxiety. Cognitions have been shown to play a major role in claustrophobia in MRI patients, thus the cognitive theoretical approach can be applied due to these interventions focusing on the educational aspect of changing irrational thoughts (Miller et al., 2011; Root, 2000; Thorpe et al., 2008). Patients were taught a variety of coping techniques to help them deal with disturbing thoughts they had when anxious, along with providing interventions that serve as distractions (Lang, Ward, & Laser, 2010; Miller et al., 2011; Root, 2000). A study conducted by Lukins et al. (1997) concluded cognitive based interventions such as breathing techniques, and guided imagery were helpful in reducing MRI patient anxiety.

A connection between cognitions and MRI anxiety was established in the early development of finding effective MRI interventions. An early case study using cognitive interventions was conducted by Klonoff et el. (1986) using a combination of progressive desensitization and relaxations techniques, which resulted in the patient being able to complete her MRI examination (Klonoff et al., 1986). This study had limitations due to the nature of being a single case study, but it laid the foundation for other studies using cognitive interventions. A later study in the mid to late 1990's was conducted using a variety of cognitive interventions including imagery techniques, music, positive affirmations and desensitizing the patient to the noise of MRI machines (Lukins et al., 1997). This study showed a significant reduction of anxiety in the experimental groups when compared to the control (Lukins et al., 1997). It should be noted that this study had one control group and two experimental groups; one received the intervention both before and during the MRI scan while the other only received an intervention before the MRI exam (Lukins et al., 1997). The results showed no significant differences in anxiety levels between the two experimental groups, which supports the idea that cognitive interventions can be effective no matter when they are employed (Lukins et al., 1997).

The two most significant cognitive based interventions in regards to anxiety in the MRI environment and cognitions and this thesis are aromatherapy and breathing techniques. Aromatherapy and breathing techniques are cognitive based interventions with immediate effects (Masaoka, Sugiyama, Katayama, Kashiwagi, & Homma, 2012). Aromatherapy can be described as a cognitive intervention, because it is a distraction technique and can help aide in changing cognitions, as scent has been shown to conjure memories (Redd et al., 1994; Zucco, Aiell, Turuani, & Koster, 2012) Breathing techniques have some of the same effects on cognitions as aromatherapy because when a person engages in controlled breathing exercises their awareness becomes focused on the act and not their emotions and thus becomes a distraction (Brown & Gerbarg, 2005a). Furthermore both of these CAM modalities have direct links to our the limbic system which governs our cognitions and they work in tandem with each other, one complementing the other in that a pleasant odor promotes deep breathing (Brown & Gerbarg, 2005a, 2012; Masaoka et al., 2012).

Aromatherapy has been one of the most popular CAM therapies used to help reduce anxiety (Hongratanaworakit, 2004; Lee et al., 2011; Perry & Perry, 2006). Aromatherapy, defined as using essential oils for therapeutic purposes either through dermal or inhalation administration (Pease & Pease, 2004). Please note all studies used in this literature review used inhaled aromatherapy as this was the route used in the pilot study. Furthermore, inhalation is believed to be the primary healing source aromatherapy has on emotions (Price & Price, 2007).

An advantage of using aromatherapy, is the immediate physiological and psychological effects it has when compared to other currently used interventions in MRI such as sedation, or hypnosis (Perry & Perry, 2006; Meledez & McCrank, 1993). Inhaled aromatherapy's immediate effect is due to the scent molecules ability to bypass the blood brain barrier a go directly to the olfactory portions of the limbic system, which regulates emotions via the autonomic nervous system (ANS) (Homma & Masaoka, 2008; Hongratanaworakit, 2004; Perry & Perry, 2006; Root, 2000). When a scent molecule enters the nasal cavity it attaches to the olfactory epithelial receptors. These receptors take this scent information and travels along the olfactory nerves and signals the limbic system via the hippocampus within the amygdala (Jimbo, Kimura, Taniguchi, Inoue, & Urakami, 2009). Once this information has reached the hippocampus the ANS is activated, neurotransmitters are secreted. The type of neurotransmitter secreted depends on which branch of the ANS is activated. If the odor is pleasant and/or attached to positive memories the parasympathetic nervous system (PNS) is activated and neurotransmitters such as GABA and serotonin are secreted producing a relaxation effect (Jimbo et al., 2009; Root, 2000; Zucco et al., 2012). In the case of an unpleasant scent and/or negative emotions attached to it the sympathetic nervous system (SNS) is activated and neurotransmitters such as norepinephrine are secreted creating anxious feelings (Jimbo et al., 2009; Root, 2000; Zucco et al., 2012). This action takes place in less than a second, approximately “300 ms-400 ms” (Homma & Masaoka, 2008, p. 1016) and reaction time may be dependent on gender with women having faster emotional reactions then men (Chen & Dalton, 2005; Perry & Perry, 2006). In short aromatherapy is the result of the scent molecule interacting with the limbic system and is dependent on the respiratory system (Homma & Masaoka, 2008; Jimbo et al., 2009).

Using a blend of essential oils may be more effective in reducing anxiety then single oils (Burns, Blamey, Ersser, Barnetson, & Lloyd, 2000). Studies have shown aromatherapy to be effective at lowering anxiety; however the majority of these used single oils in their interventions creating a gap in the literature (Burns et al., 2000). Several single essential oils have been studied in reducing to including, but not limited to lavender, rosemary, bergamot, ylang ylang, rosemary, melissa, valerian, roman chamomile, neroli, cedarwood, orange oil, eucalyptus, rose, jasmine, and vetiver (Gorji, Koulivand, & Ghadiri, 2013; Lee et al., 2011; Moss, Cook, Wesnes, & Duckett, 2003; Saiyudthong & Marsden, 15 2011). While some studies have used essential oil blends in a variety of medical applications there have not been any evaluating its use in the MRI environment, and this pilot study fill that gap (Jimbo et al., 2009; Price & Price, 2007).

Aromatherapy has been used to help reduce anxiety in a variety of medical settings. There have been several studies evaluating its use in reducing anxiety dental, post-surgical, and postpartum anxiety with positive result (Braden, Reichow, & Halm, 2009; Lee et al., 2011; Lehrner, Marwinski, Lehr, Johren, & Deecke, 2005). A pilot study by Conrad & Adams (2012) evaluating the effects lavender on anxiety and depression of postpartum women found a significant reduction of anxiety in the experimental using the aromatherapy. Although this study showed positive results, only women were used, which limited the verifiable effect to women (Conrad & Adams, 2012). Furthermore this study was conducted over a four week period and when applying aromatherapy to the MRI environment these results cannot be compared, as MRI anxiety is usually acute (Conrad & Adams, 2012). Another study reviewing the effect lavender had on pain and anxiety of post-operative patients had an equal number of male and female participants, and showed a significant reduction of both pain and anxiety supporting the effectiveness of aromatherapy for both genders (Braden et al., 2009).

Other studies demonstrated various medical procedures aromatherapy has been used, included using essential oils to reduce dental phobia in children. This study concluded aromatherapy helped reduce dental procedure fears in children (Jafarzadeh, Arman, & Pour, 2013). Another study assessing the effect vaporized orange oil had on dental patients showed increase activity of the parasympathetic nervous (PNS), and promoted relaxation by 12% while decreasing the sympathetic nervous system (SNS) by 16% and showed a significant reduction on anxiety in adult dental patients (Jafarzadeh et al., 2013; Lehrner et al., 2005; Perry & Perry, 2006). While this study was conducted on both men and women, anxiety reduction was more pronounced in women (Lehrner et al., 2005).

A systematic literature review of studies that used aromatherapy to reduce anxiety over a twenty year period was conducted. Out of the 14 studies reviewed six (6) used inhaled aromatherapy as an intervention with one using music therapy as well (Lee et al., 2011). Five of the six studies showed aromatherapy to be effective in reducing anxiety and one showing no significant change (Lee et al., 2011). These studies included participants that experienced anxiety for a variety of reasons, with the majority undergoing medical treatments and a diverse group of individuals. This literature review concluded aromatherapy could in fact be used to help reduce the symptoms of anxiety in a variety of medical environments (Lee et al., 2011).

The use of scent has been evaluated in the MRI environment to help reduce anxiety and consequently the number of canceled exams with varying results (Redd et al., 1994). Aromatherapy was reported to help treat anxiety, including cognitive and stress related disorders (Perry & Perry, 2006; Conrad & Adams, 2012). This directly correlates with MRI claustrophobia, as it is a stress related disorder in which cognitions greatly contributes to anxiety during the exam (Lukins et al., 1997; Starcevic, 2004; Thorpe et al., 2008).

The use of fragrance has been successfully used to reduce anxiety in the MRI environment (Redd et al., 1994). Two studies dealing specifically with using scents in the MRI environment was found. The earlier study conducted by Redd et al (1994) showed a reduction in overall MRI anxiety by 63% in patients using a vanilla scent administered through a nasal cannula (Redd et al., 1994). This study went one step further and evaluated the experimental group participants who considered the scent pleasing and their level of anxiety and found a noteworthy reduction in those who found the aroma pleasant (Redd et al., 1994). This study hypothesized fragrance had a cognitive effect on patients enabling them to remain calm during their examination. This was in alignment with a study that concluded scents can affect a person's mood and consequently their cognitions (Perry & Perry, 2006; Redd et al., 1994). In essence the scent may have blocked anxious feelings and replaced them with more positive ones, and supports the theoretical foundation of using cognitions in reducing MRI anxiety (Redd et al., 1994). Furthermore a pleasant scent causes people to slow their breath and deepen their inhalations triggering a relaxation response (Moore, Hickson, & Stacks, 2010). This same study noted possibly decreasing anxiety further in MRI patients by allowing them choose their own scent and adding relaxations techniques (Redd et al., 1994). This pilot study used this suggestion and allowed patients to choose their own scent and incorporated breathing exercises as an additional relaxation technique. Lastly this study concluded that while positive results were shown in using scent to reduce MRI anxiety, others studies reproducing its results will be necessary before using fragrance can be include as a standard of care in controlling MRI anxiety, and was aim of this thesis (Redd et al., 1994).

Another study conducted in Germany by Schellhammer et al. (2013) evaluated the effect scent had on reducing motion artifacts, which has been closely linked to patients experiencing anxiety. Although some participants said the scents created a “positive mood” (Schellhammer et al., 2013, p. 795) the study did not show the use of scent to have a significant effect on motion artifacts or anxiety levels in patients (Schellhammer et al., 2013).

There may have been several reasons the Schellhammer study was not able to get similar results to the Redd et al (1994) study. The Schellhammer (2013) study did not use the same scents and the administration of scents was different (Redd et al., 1994; Schellhammer et al., 2013). When comparing the methods of scent administration in these studies, the Redd et al. (1997) study was diligent about not overwhelming the olfactory senses, by administering a low (5%) concentration of the scent in 30 second spurts, and alternating it with 60 seconds of unscented air. This may have helped the effectiveness of the scent, as the longer a person is exposed to the aroma the less effective it becomes (Price & Price, 2007). Comparatively the Schellhammer study took great care in ensuring the scent was concentrated in the bore of the machine, but it was continuously administered possibly reducing its effectiveness (Schellhammer et al., 2013).

Neither of these studies used true aromatherapy, leaving a gap in the use of essential oils in the MRI environment. The scents used in both studies were synthetic and in order to be truly considered aromatherapy essential oils must be used (Price & Price, 2007; Redd et al., 1994; Schellhammer et al., 2013). The scents used in the Schellhammer (2013) study were commercial synthetic fragrances commonly used in tanning beds and elevators, while the Redd (1997) study used synthetic vanilla scent. While both of these studies contributed to the literature on scent use in the MRI environment, a gap of using real aromatherapy was necessary.

In conclusion there is a need for further research on using aromatherapy in the MRI environment. Scent has helped patients with anxiety in a variety of medical procedures and treatments with varying results (Braden et al., 2009; Jafarzadeh et al., 2013; Jimbo et al., 2009; Price & Price, 2007; Redd et al., 1994; Schellhammer et al., 2013). Yet it is clear from the contrasting results of the studies that specifically evaluated it MRI anxiety further research is necessary.

Scent can increase a customer's sense of satisfaction (Goldkuhl & Styvén, 2007). Scents have been shown to elicit strong emotional reactions with anxious individuals being more responsive to pleasant aromas, making it a feasible way of decreasing anxiety in MRI patients and increasing patient satisfaction (Chen & Dalton, 2005; Goldkuhl & Styvén, 2007; Zucco et al., 2012). As stated earlier, the purpose of studying this phenomenon was to create a customer service protocol to aide in creating a more positive experience for MRI patients. The benefits scent can have on influencing customer service was seen in a paper evaluating how aroma can be used as a marketing tool and a way of enabling a service to be more palpable (Goldkuhl & Styvén, 2007). This relates to MRI as it is a service provided to patients, thus incorporating aromatherapy in imaging centers helps patients create positive associations with the exam. Furthermore, MRI centers that use scent may create a competitive advantage over other imaging centers (Goldkuhl & Styvén, 2007).

There are two types of breathing, metabolic and behavioral (Homma & Masaoka, 2008). Behavioral breathing is controlled by the individual and can be influence by both internal and external factors (Homma & Masaoka, 2008). The individual has no control over metabolic breathing, which is managed by the autonomic nervous system (Homma & Masaoka, 2008). Although emotions are governed by metabolic and behavioral breathing, behavioral breathing is the basis of breathing techniques which will be discussed in further detail in this section and their relationship with breathing.

Breathing techniques, defined as eliciting a relaxation response through conscious breathing have been found to be beneficial in reducing anxiety and was cited as a popular and effective self-regulation technique by MRI patients (Brown & Gerbarg, 2005; Lang, Ward, & Laser, 2010). This relaxation response is elicited through the autonomic nervous system that consists of two parts, the parasympathetic nervous system (PNS) and sympathetic nervous system (SNS), and are responsible for the relaxation and stress responses respectively (Brown & Gerbarg, 2012; Fried & Grimaldi, 1993). The autonomic nervous system and neuroendocrine functions that control stress and relaxation responses can be positively influenced by controlled breathing (Brown & Gerbarg, 2005). One study suggested the nervous system response can be activated in less than five (5) minutes to help achieve a more relaxed state making breathing techniques a viable way of dealing with anxiety in the fast paced MRI environment (Brown & Gerbarg, 2012).

Breathing techniques reduces stress by increasing the amount of oxygen in the body while decreasing carbon dioxide (Brown & Gerbarg, 2012; Fried & Grimaldi, 1993). The reduction of carbon dioxide is the basic reason breathing techniques are effective in activating the relaxation response (Brown & Gerbarg, 2012). An excessive amount of carbon dioxide in the blood creates lactic acidosis which aggravates symptoms of panic and anxiety causing the patient to hyperventilate (Fried & Grimaldi, 1993).

Hyperventilation is a breathing disorder associated with anxiety disorders commonly seen in the MRI department, such as claustrophobia and panic attacks. When a person suddenly feels anxious there is a decrease in the amount of blood reaching the brain (Fried & Grimaldi, 1993). This reduction of oxygen to the brain causes neurocirculatory asthenia or air hunger (Fried & Grimaldi, 1993; Giardino, Friedman, & Dager, 2007). Air hunger, also described as breathlessness, is when a person feels as though they are not getting enough air, causing them to breathe faster, setting the stage for hyperventilation (Fried & Grimaldi, 1993). It should be noted that it is not clear whether hyperventilation is the cause of anxiety or if anxiety causes it (Fried & Grimaldi, 1993). A study by Giardino et al. (2007) reviewing the effect anxiety had on blood flow to the brain using functional MRI (fMRI), found the MRI exam itself caused anxiety and a reduction in blood flow to the brain was observed.

Breathing techniques that incorporate slow breathing along with resistant breathing helps anxiety disorders in two ways. Firstly, it increases the heart rate variability (HRV), which aides in activating the PNS's relaxation response (Fried & Grimaldi, 1993). Secondly it decreases the amount of carbon dioxide by increasing the number of alveoli available to receive oxygen (Brown & Gerbarg, 2005b; Fried & Grimaldi, 1993). Lastly, breathing techniques increases communication between the brain structures of the limbic system strengthening its ability to regulate emotions (Brown & Gerbarg, 2012).

Breathing techniques were found to decrease both physiological and psychological arousal during threatening situations, as MRI exams are sometimes viewed as such a situation (Cappo & Holmes, 1984; Harris et al., 1999; Thorpe et al., 2008). A study showed consistent positive results in reducing anxiety using the breathing techniques (Cappo & Holmes, 1984). This study evaluated a combination of breathing patterns to included; inhaling slowly and exhaling quickly, exhaling and inhaling at equal intervals and finally inhaling quickly and exhaling slowly (Cappo & Holmes, 1984). This study concluded inhaling quickly and then exhaling slowly was more effective than the other breathing combinations (Cappo & Holmes, 1984).

Breathing techniques have been shown to be effective in treating specific psychological disorders (Meuret, Wilhelm, Ritz, & Roth, 2003). Two studies have shown breathing techniques aided in reducing symptoms associated with panic attacks and anxiety, which are often seen in MRI patients (Brennan et al., 1988; Brown & Gerbarg, 2005b; Grey et al., 2000; Meuret et al., 2003). Furthermore studies have shown brief breathing training beneficial in reducing the frequency and severity of panic attacks sufferers (Brown & Gerbarg, 2005b; Meuret et al., 2003). Breathing techniques are also believed to give a greater sense of control over the physical symptoms and catastrophic cognitions associated with panic attacks and anxiety, which are major contributing factors to MRI anxiety (Meuret et al., 2003; Tornqvist et al., 2006).

There were several studies that evaluated MRI anxiety and mentioned breathing techniques was used by patients themselves to self-regulate their emotions, yet no studies focused solely on breathing as an intervention (Munn & Jordan, 2013; Murphy & Brunberg, 1997; Quirk et al., 1989b). Although none of these studies used breathing techniques specifically as an intervention, a qualitative study conducted by Tornqvist et al. (2006) concluded one of the major factors effecting anxious MRI patients was a lack of self-control, which breathing techniques may help patients regain (Meuret et al., 2003). This same study found a common complaint of patients experiencing shortness of breath while in the MRI machine (Tornqvist et al., 2006). With that said patients having the ability to control their breath is an important component in helping them regain self-control, and reducing shortness of breath.

Another study reviewing the effect desensitization has on an anxious MRI patient used breathing technique, amongst others as cognitive interventions to help a patient complete her examination (Klonoff et al., 1986). Furthermore a study evaluated interventions patients used naturally to help reduce their stress during their MRI (Murphy & Brunberg, 1997). Out of all the impromptu interventions 11% of patients used breathing techniques to help them relax during their exam (Murphy & Brunberg, 1997).

A number of studies have shown the calming physiological and psychological attributes breathing techniques can produce (Brown & Gerbarg, 2005a; Cappo & Holmes, 1984; Thorpe et al., 2008). It has been shown to help treat the anxiety related disorders seen in the MRI environment (Brown & Gerbarg, 2005a; Meuret et al., 2003; Thorpe et al., 2008). While there have been MRI anxiety studies that have mentioned it as an intervention, there is still a significant gap in research that focuses solely on breathing techniques, which this current thesis study fills.

Imagery techniques described as methods of creating mental images to create an altered state of consciousness (Spiegel & Moore, 1997). Two types of imagery techniques discussed in this section are guided imagery (GI) and hypnosis. Hypnosis and GI are cognitive based therapies that use the power of suggestions and thoughts to create positive mental images to help promote relaxation and healing (Cooper & Stollings, 2010; Spiegel & Moore, 1997).

Guided imagery has been shown to help reduce anxiety (Cooper & Stollings, 2010; Spiegel & Moore, 1997). This cognitive based therapy has been shown to be effective when practiced for 15 minutes or under making it a quick and valuable intervention (Cooper & Stollings, 2010). A study by Thompson & Coppens, (1994) that evaluated guided imagery as an MRI anxiety intervention. They hypothesized patients who received guided imagery before and during their MRI examination would have a less anxiety and a reduced amount of movement during their test (Thompson & Coppens, 1994). Results showed a reduction of anxiety and movement in the experimental group, while no decrease was observed in the control group (Thompson & Coppens, 1994). Limitations were noted by the study's authors concerning patient movement due to patients possibly not revealing they moved because of being instructed not to move (Thompson & Coppens, 1994). A study by Quirk et al. (1989) used guided imagery in a study assessing three psychological interventions for MRI claustrophobia. The guided imagery arm of this study showed significantly less anxiety then the other two, which were written information and counseling with written information (Quirk et al., 1989b). Furthermore guided imagery was implemented for 12 minutes supporting the fast efficient implementation of this treatment (Cooper & Stollings, 2010).

Hypnosis has been successfully used to reduce anxiety in a number of medical situations and included its use in the MRI environment and a surgery suite (Cooper & Stollings, 2010; Tusek et al., 1997). A study by Tusek et al. (1997) showed post-surgical colorectal patients experience not only less anxiety and pain, and needed half as much pain medication then the control group.

In the MRI environment hypnosis was used successfully to reduce anxiety in two cases (Friday & Kubal, 1990; Lang et al., 2010). The first study looked at the effect hypnosis and rapport training of technologists and support staff had on reducing the number of cancelled MRI examinations due to nervousness (Friday & Kubal, 1990; Lang et al., 2010). This study concluded that hypnosis and staff training was effective in reducing cancelled MRI exams (Lang et al., 2010). There were limitations in this study. Firstly the study ended prematurely due to changes in the study site's organizational structure (Lang et al., 2010). Secondly the study stated patients could be put into a hypnotic state in less than 12 minutes, yet the study does not mention how long it took to accomplish this in their methods section. Lastly the hypnosis training for technologist required 20 hours of training, which is a considerable amount of training (Lang et al., 2010).

A second study conducted by Friday & Kubal (1990) showed hypnosis helped ten patients complete their MRI examination. Like the study mention above there were study limitations. The hypnosis required extensive training of personal and took 45 minutes to place patients in a hypnotic state (Friday & Kubal, 1990). Furthermore out of the 10 patients who were able to complete the exam two had taken a sedative (Friday & Kubal, 1990). In essence, although hypnosis is an effective intervention for anxious MRI patients it may not be practical in the MRI environment due to requiring substantial training and implementation time.

Throughout history music has been recognized to have positive effects in reducing patient anxiety and promote healing (Nilsson, 2008). In 1914 music was used as a distraction technique for his patients undergoing surgery (Nilsson, 2008). Currently music therapy has been “recognized as an established allied health profession” (Kemper & Danhauer, 2005, p. 283) with its own credentialing body, The American Musical Therapy Association (Kemper & Danhauer, 2005).

Music has been used as a tool to help patients reduce emotional stress in order to better tolerate stressful medical procedures (Bally, Campbell, & Chesnick, 2003; Kemper & Danhauer, 2005). Music has been known to positively affect a person physiologically and psychologically making it a cognitive based intervention (Voss et al., 2004). When patients are stressed, physiological changes occur including an increase in blood pressure, heart rate and levels of cortisol, and music has been shown to effectively decrease all of these symptoms (Bally et al., 2003). When these biological changes occur they also change a person on a “psychologically and social emotionally” (Kemper & Danhauer, 2005, p. 283).

Music has been used to help pre-operative and post-operative patients, reduce anxiety before their surgeries (Cunningham, Monson, & Bookbinder, 1997; Voss et al., 2004). Cunningham et al. (1997) conducted a study using music for pre-operative plastic surgery patients. Each patient listened to 30 minutes of classical music, and afterwards reported feeling “calmer and more relaxed” (Cunningham et al., 1997, p. 677). Additionally the patient's blood pressure and heart rates were reduced confirming a less anxious state (Cunningham et al., 1997).

Another study by Voss et al., (2004) compared the difference between anxiety and pain levels of post-operative open heart surgery patients during chair rest. This study concluded patients who received relaxing music had a significant reduction in both pain and anxiety, while the non-treatment group had no noteworthy changes (Voss et al., 2004). Another study evaluated the effects music had on oncology patients and concluded it made them less anxious (Kemper & Danhauer, 2005). Patients reported the effect of the music as “relaxed and uplifted”, “soothing and relaxing”, “calmed my nerves” and “felt calm and happy” (Kemper & Danhauer, 2005, p. 284). As these studies demonstrated music can help reduce anxiety in medical settings and has helped MRI patients as well.

Although music has been shown to be effective in reducing anxiety in patients undergoing other medical procedures there were a limited number of studies found specifically on its use for MRI anxiety (Phillips & Deary, 1995). Music has been a commonly used cognitive intervention in the MRI environment to reduce anxiety in patients (Phillips & Deary, 1995; Walworth, 2010).

Two studies were found on using music in the MRI environment. The first looked at the difference in how MRI exams were perceived and anxiety levels of patients given live music, compared to patient selected recorded music (Walworth, 2010). Results showed patient's perception of MRI improved and anxiety levels of patients who received live music were reduced compared to the pre-recorded music intervention group (Walworth, 2010). This study also found the live music group spent less time in the MRI machine allowing more patients appointment slots and was interpreted as a cost saving measure (Walworth, 2010). Although this study supported music can an effective cognitive intervention in to reduce anxiety, it is highly impractical to have live music in the MRI room as it is a restricted area and being able to offer live music on a regular basis is highly unlikely. Therefore this study has considerable limitations due to its impracticality. This study also mentioned the cost savings of live music in the MRI department, but failed to mention the costs associated with paying the musicians (Walworth, 2010). Additionally this study was dependent on having a music therapist on hand to ‘prescribe’ the most effective music to each patient (Walworth, 2010). Again it is very unlikely an imaging department would employ a music therapist solely for this purpose and any cost savings from reduced scan times would be absorbed by the music therapist and musicians.

A second study by Grey et al., (2000) used the volume of music as an MRI intervention combined with other anxiety reducing. Patients were given a button to inform the technologist they wanted a change in the volume of music (Grey et al., 2000). This study found a significant reduction of anxiety in the experimental patients, but there were no specific mention of how music volume played a role in anxiety reduction creating a study limitation (Grey et al., 2000).

Although music has been used successfully to help reduce anxiety in MRI patients, noise from the MRI machine may reduce the effectives of this intervention, because music does not completely cancel out the machine's noise (McNulty & McNulty, 2009). This is an important factor, because a study conducted by McNulty & McNulty (2009) that looked specifically at the negative effects MRI machine, found noise to be a significant factor in patient anxiety.

Neurolinguistic programming is another cognitive intervention and was evaluated as an MRI anxiety intervention. Neurolinguistic programming is a cognitive therapy used for people who have nonspecific phobias (Bigley et al., 2010). This technique's goal was to replace negative associations with MRI exams with more positive ones (Bigley et al., 2010). The study evaluated neurolinguistic programming as an alternative to using general anesthesia in patients who were not able to complete their MRI exam (Bigley et al., 2010). Study results showed a 76% success rate using neurolinguistic programming for patients who had previously failed to complete their MRI (Bigley et al., 2010).

That being said this cognitive intervention has several limitations, which included requiring one hour to perform the technique (Bigley et al., 2010). Furthermore this technique required a considerable amount of specialized training for technologists, involving 20 days of training over a six month period (Bigley et al., 2010).

As research revealed there are a variety of Non-CAM and CAM interventions that have been used to help reduce anxiety in MRI patients, but more intervention are necessary. There has been a plethora of interventions employed to combat MRI anxiety, with more needed that focuses on improving the patients experience and the patient technologist relationship (Redd et al., 1994; Tischler et al., 2008; Tornqvist et al., 2006). A survey of experienced MRI technologists concluded that while many interventions have been employed, including cognitive techniques along with utilizing shorter and wider bores, patient education, pre-scan visits and the use of music, anxiety is still a significant problem (Munn & Jordan, 2011; Redd et al., 1994; Spouse & Gedroyc, 2000; Tischler et al., 2008).

Some studies suggest combining interventions may be more effective than using them individually (Klonoff et al., 1986; Quirk, Letendre, Ciottone, & Lingley, 1989a; Redd et al., 1994). While others studies have combined different interventions such as the use of written information with cogitative approaches, and the use of hypnosis with technologists rapport training, there have not been any evaluating a combination of breathing techniques and aromatherapy (Lang et al., 2010; Quirk et al., 1989). Therefore, the current study aim was to add to MRI anxiety literature by evaluating the effectiveness of these two cognitive based CAM modalities, which may ultimately contribute to reducing cancelled MRI exams due anxiety.

### DETAILED DESCRIPTION OF PREFERRED EMBODIMENTS

Certain preferred embodiments of a method according to the present invention include the following treatment steps, which can be used in sequence, alone, or in any combination, or in any order. Preferably, the treatment includes: Magnetic Resonance Imaging (MRI) Breathing Techniques and Aromatherapy Protocols (SIGBA).

In one embodiment of the present invention, an MRI intervention was created by combining breathing techniques and aromatherapy (SIGBA) and tested in a mixed methods quasi-experimental pilot study. A 76.5% statistically significant reduction in anxiety in MRI patients was observed. Ultimately this research contributes to advancing the MRI profession by creating a customer service protocol for training technologists on how to effectively deal with anxious patients using these CAM modalities. It also adds an additional intervention for MRI anxiety and an alternative to patients that may not want or cannot use sedation. This research contributes to the literature of CAM usage and integration into conventional (or orthodox) medicine. It also contributes to CAM usage in the MRI environment, specifically using scent and breathing techniques as cognitive interventions. Other implications include cost savings for patients and medical facilities from fewer cancelled MRI exams. Most importantly, this research increases the quality of patient care in the MRI environment.

The study design used was concurrent triangulation mixed methods, emphasizing quantitative data with qualitative data used to inform the quantitative, in evaluating the role complementary alternative medicine (CAM) modalities, aromatherapy and breathing techniques have in reducing anxiety and consequently decreasing cancelled MRI exams due to anxiety. Triangulation strategy of data was used, because it is a well validated approach and one of the most familiar strategies used in mixed methods research (Creswell, Klassen, Clark, & Smith, 2011). A mixed method framework created a more holistic approach to exploring possible solutions to the complex issue of cancelled MRI exams due to anxiety (Creswell, 2009). Furthermore this framework helped gain more insight into the issue than exclusive use of qualitative or quantitative methods alone (Creswell, 2009).

This invention shows how complementary and/or alternative medicine modalities, such as aromatherapy and breathing techniques, can be effective in reducing anxiety and reestablishing emotional and physical homeostasis in patients undergoing MRI examinations. The research supporting this invention includes original research from a pilot study testing the effectiveness of combining breathing techniques with aromatherapy and tested the following hypotheses: 1. Combining aromatherapy and breathing techniques decreases anxiety in MRI patients. 2. A combination of breathing techniques and aromatherapy is more effective than using aromatherapy alone. 3. Breathing techniques and aromatherapy reduces the time it takes medication to take effect in patients whose medication had not reached maximum effectiveness before their appointment. Ultimately this research contributes to creating a customer service protocol for training MRI technologists on how to effectively deal with anxious patients using these CAM modalities. This customer service training will also incorporate evidence based solutions for identifying potentially anxious patients and applying CAM and cognitive restructuring techniques to ease their fear. Furthermore the pilot study results and thesis subtopics will contribute to incorporating CAM into MRI technologist training protocol, and provide support for integrating CAM into medical imaging.

The participants were acquired at Rebound Orthopedics and Neurosurgery. All participants were recruited at their office located in Portland, Oreg. except for one who was recruited from their office if Vancouver, Wash. A nonprobability or quasi experiment convenience sampling was used for participant recruitment. Quasi experimental sampling was used because randomization was not possible and the limited number of available participants, although unlike most study designs of this type a control group was included to strengthen the study (Cottrell & McKenzie, 2005).

The participant population sampling for the pilot study included those fluent in English, 18 years of age or older. There were exclusions of participants based on qualifier questions described later in the procedure section. The study was limited to the following participants, because the study focus was to evaluate the effectiveness of CAM interventions for claustrophobic/anxious MRI patients. A key requirement for recruitment of participants was based on the type of exam they were scheduled to undergo, and required the majority of their body being in the MRI scanner. This included all exams from the hip region and above. The MRI exams performed in this study included the following; eight (8) cervical spines, 11 lumbar spines, with two being with and without contrast, all in which were performed with patients positioned feet first, 11 shoulders, two 15 (2) elbows, one (1) brain with and without contrast, and one (1) pelvis and wrist respectively. The reasoning for this sampling was that patients whose head is outside of the MRI machine do not suffer from the same degree of anxiety as those with their head inside the magnet (Murphy & Brunberg, 1997). Sampling of participants prescribed medication specifically to reduce anxiety during their MRI exam, as well as those use that did not use medication were included. Both of these populations were chosen because it was hypothesized that CAM modalities, breathing techniques and aromatherapy would reduce the time it took to their medication to take effect in patients whose medication was not effective before the start of their exam. Lastly, self-described claustrophobic patients who either verbally expressed or answered yes to the question “Do you suffer from claustrophobia?” on the MRI scheduling form were included in the sampling.

A total of 43 potential participants were invited to the study. There were eight (8) who refused to participated for several reasons, such as time constraints, a lack of anxious feelings, and simply wanting to complete the exam without any additional disruptions. The total number of actual participants was 38. These participants were divided into four arms of the study with two arms in the experimental group and two in the control group. The experimental arms included one with participants who used anti-anxiety medication and the other without drugs. The medicated experimental arm had a total of five (5), while the non-medicated arm had 13. Likewise the control arms included participants who used anti-anxiety medication and the other without. The control medicated group had eight (8) total and the non-medicated arm 12.

The demographics of participants were as follows. There were 19 females and 16 males, with 32 Caucasian and three (3) African Americans, in which two (2) were male and one (1) female. The age ranged was 30 to 73 years old with an average age of 57.7 years.

The physiological data collected included heart and respiratory rates of both study groups. The physiological data was collected using peripheral gating that measured the participants heart rate using a pulse oximeter type apparatus and breathing bellows, which are standard, features on MRI machines. These features are traditionally used to monitor patient's heart and breathing rates while undergoing specialized MRI imaging techniques in which these physiological readings are gated or monitored in order to reduce blurring of images from breathing and heart motion. Fortunately these features can be use independently to accurately record the heart and respiratory rates, yet they should not be used in lieu of monitors specifically calibrated to measure these physiological parameters. The heart rate was collected by the MRI machine using a pulse oxygen sensor place on the patient's index finger, while the patient's respiratory rate will be recorded using breathing bellows wrapped around the patient's abdomen at the highest point of inspiration.

The qualitative and quantitative data was collected via a questionnaire that utilized a combination of Likert scale questions/statements along with closed and open ended questions. The quantitative data was collected, included one multiple choice question, and a combination of closed ended questions or statements utilizing a 7-point Likert scales. Likert scales were chosen for the questionnaire in part to them being well validated, reliable, and they were used in a study conducted by Redd et al. 1995 that evaluated the use of scent for MRI anxiety, in which this study is loosely based on (Alwin, 1997). Furthermore several questions regarding anxiety and fragrance were duplicated from the study mentioned above.

The Likert scale questions included were as follows; pre and post exam questions of “I currently feel” with the anchors “not anxious at all” and “extremely anxious”. The post exam Likert scale questions with the same anchors for the experimental group and control group focused on the participants' anxiety level included “what was your average, most, and least level of anxiety while in the MRI machine?” The experimental group received an extra question regarding the interventions that stated “what was your anxiety level after the breathing exercises and aromatherapy?” with the same anchors above.

Questions focused on aromatherapy were included for both study groups, because the control groups received sham aromatherapy, and will be discussed further. The questions pertaining to the aromatherapy had the following questions and anchors. “how relaxing was the fragrance?” and had the anchors, “not at all relaxing” to extremely relaxing.” The patients could also rank the intensity of fragrance from “not at all intense” to “extremely intense.” The experimental group had an additional question about the pleasantness of their chosen scent, with anchors “unpleasant” to “pleasant.” Participants that took anti-anxiety medication had two additional questions. The control group questions were “the aromatherapy increased the relaxation effect of my medication” with anchors strongly “agree” to “strongly disagree.” The statement “the medication alone was very effective in reducing my anxiety” with anchors, “strongly agree” to “strongly disagree” was proposed to both medicated control and experimental groups. Only the medicated experimental group had the following statement “the aromatherapy and breathing exercises increased the relaxation effect of my medication.” with anchors “strongly agree” to “strongly disagree.”

There were several post exam non-Likert scale questions included on the questionnaire for both study groups which included open ended qualitative questions and a multiple choice question. The multiple choice question was included to help corroborate or inform the answers from the Likert scale questions and statements about the interventions. The multiple choice question was, “which was most effective in reducing your anxiety today?” The experimental choices were Aromatherapy, Breathing Exercises, Both Equally and Neither, while the control group's choices were Aromatherapy (sham), MRI Information Sheet, Both Equally and Neither. The qualitative open ended questions asked the participants to describe their overall MRI experience in their own words. The experimental questions pertained to, aromatherapy, breathing techniques, and anxiety and the control group's questions focused on aromatherapy, MRI information sheet and anxiety. Lastly both groups were asked to make suggestions of how to make the MRI experience more comfortable for patients.

The recruitment process took place after the patient's exams were scheduled with some recruitment taking place the day of their MRI exam. This process included identifying patients who answered yes to being claustrophobic on the MRI scheduling form, and scheduled during the time the primary investigator was performing MRI examinations. These patients were called the night before their appointments and invited to participate in the study using a pre-written script explaining the research study. If patients agreed to participate in the study they were instructed to arrive thirty (30) minutes before their exam to fill out the required paperwork. Once the phoned participants arrived for their appointment they were given a consent form to read and sign along with the appropriated questionnaire. If patients did not answer the call a message was left inviting them to join the study and if they were interested in participating to arrive (30) thirty minutes before their appointment time and inform the technologist of their interest. If participants showed interest in the study the same day recruitment procedure as described in section below.

The recruitment process the day of the patient's MRI exam was designed for three different types of potential participants with the procedure being the same for all three. The first type were those who were not able to be contacted by phone, same day scheduled MRI exams, and lastly patients who verbalized being anxious or claustrophobic to the technologist before their exam. If participants told the primary investigator they were anxious or suffered from claustrophobia they were invited to participate in the study. If they showed interest, they were given a same day recruitment form with qualifier questions along with an explanation of the study and asked to read it. If the participant agreed to participate they were given a consent form to sign and appropriated questionnaire administered. Afterwards the research proceeded for the experimental and/or control group as described in the procedure section of this chapter. Please note that if the patient answered yes to any questions on the qualifier questionnaire they were automatically assigned to the control group to avoid any negative reaction from the intervention. The only exception to this was if they answered yes to being diagnosed with an anxiety disorder. In this case they were disqualified from participating in the study and were provided with usual care.

The intervention procedures for the control and experimental groups are described in this section and were as follows. Please note that the procedure for experimental groups arms, with and without medication were the same, while the control group arms with and without medication were the same as well.

The procedure for experimental participants after consent was obtained and pre-exam questions completed was as follows. Each participant was given the normal instructions of how to prepare for their MRI exam. This included changing into a gown and sweat pants or shorts and removing all jewelry and hair accessories. Once the participant was ready and placed on the MRI scanning table the breathing bellows was place on their stomach and pulse oximeter on an index finger. After insuring good waveforms were produced the pre-scan physiological data was recorded on the Physiological Data Sheet. The patient chooses the scent they would like to use for their aromatherapy. There were four options available Fruity, Floral, Earthy, and Minty. Two (2) drops of the selected aromatherapy oil blend was then place on 2×2 inch sterile gauze and taped inside the MRI machine bore in front of the air vent with the fan setting on low.

The procedure for the control arms of the study were as follows. Once consent was obtained participants were asked to read a patient information sheet produced by the American Society of Radiologic Technologists (ASRT). This sheet included an explanation of what an MRI exam was, how to prepare for it, what to expect during the exam and post exam information. After reading the MRI information sheet participants were asked to answer pre-exam questions on the Control Group Questionnaire. Once the questionnaire was completed they were asked to dress for the exam as described above. Once the participant was in the MRI room and on the exam table they were hooked up to the physiological monitors as describe above in the experiments procedure section. Once the monitors were in place pre-exam physiological readings were recorded on the Control Physiological Data sheet. The patient was then told they would be receiving aromatherapy. The PI then placed two drops of the sham aromatherapy (grape seed oil) on a 2×2 piece of sterile gauze and placed in the back of the MRI machine in front of the vent with the fan on low. The PI insured the sham aromatherapy was placed on the gauze in plain view of the patient along with placing it in front of the machine vent along with verbalizing its use during the exam.

Grape seed oil is a common carrier oil in aromatherapy (Price & Price, 2007; Workwood, 1990). A carrier oil is an oil in which the essential oils are diluted and used to “carry” them to their intended administration (Price & Price, 2007). Grape seed oil was used for the sham because of its inert characteristics, such as it being tasteless and nearly odorless (Price & Price, 2007). Additionally, when considering other possible carrier oils, grape seed oil only had one therapeutic indication for eczema, which would only be applied if administered transdermal and was not the case for this pilot study (Price & Price, 2007). Thus this carrier oil was less likely to produce any type of reaction from inhalation by the study participants, while still appearing similar to the essential oils. No other instructions or information concerning the sham aromatherapy was given to the participant and the exam proceeded as described next. The exam was explained to the participant, and they were given the emergency call button and reminded that they have control over the situation and could always squeeze the emergency button to be let out of the machine at any time. They were also informed that the PI would be checking in on them throughout the exam informing them of each sequences length. During the halfway point of the exam physiological data was collected. Once the exam was complete the patient was told to remain still once out of the MRI machine for accurate physiological readings. The readings were taken and once the participants were dressed the questionnaire was returned to them for post exam completion.

The patient has a choice of four different aromatherapy scent blends. All of the blends include pure undiluted essential oils with the exception of vanilla, which was a CO₂ extract, and administered inactively through inhalation. The blends include; #1 a fruity, #2 floral, #3 earthy, and #4 minty. The essential oils used include bergamot, lemongrass, spearmint, rosemary, cardamom, oil of orange, vetiver, geranium, lavender, patchouli, sandalwood, palmarosa, roman chamomile, pettigraine, neroli, vanilla, ylang ylang and marjoram.

The following are formulations of each aromatherapy blend; these blends are approximately one (1) fluid ounce each. These are the quantities as they were used in the study. The origins of the aromatherapy scents are provided in parentheses. In other embodiments of the invention or formulations, the scents have different origins.

Fruity Blend (Approx.) 1.1OZ: Orange 5 fold (USA)=9.5 ml, Lemongrass (Indian)=5 ml, Vanilla CO₂=8.5 ml, Neroli (Egyptian)=2.5 ml, Bergamot (Italian)=5 ml, and Pettigraine (Paraguay)=2.5 ml.

Floral Blend (Approx.) 1OZ, Total of 30.08 ml: Palmarosa (India)=7.5 ml, Geranium (Egypt)=5.65 ml, Roman Chamomile (Oregon)=1.87 ml, Lavender (Oregon)=3.76 ml, Ylang Ylang 2nd (Madagascar)=5.65 ml, and Vanilla CO₂=5.65 mi.

Earthy Blend (Approx.) 1OZ: Patchouli Dark Aged (Indonesia)=5.25 ml, Sandalwood Mysore (India)=2.6 ml, Vanilla CO₂=1.05 ml, Lemongrass (India)=1.6 ml, Cardamom Whole (India)=1.6 ml, Orange 5 Fold (USA)=1.6 ml, Vetiver (Indonesia)=3.7 ml, Geranium (Egypt)=10.5 ml, and Bergamot (Italian)=2.1 ml.

Minty Blend (Approx.) 1OZ: Rosemary Verbenone (USA) 5 ml, Spearmint (Oregon) 12.5 ml, and Marjoram Sweet (Hungary) 12.5 ml.

To begin the patient is placed on the MRI scanning table and hooked up to the breathing bellows (If available). It should be noted that the breathing bellows may be used to monitor the patient's breathing and can help indicate the patient's emotional state. The patient chooses the scent they would like to use for their aromatherapy. Place (2) drops of the patient chosen aromatherapy oil on a 2×2 gauze and placed in front of an air vent (using tape, if needed) located inside the MRI machine bore. Set the fan to low, if possible. Once the patient confirms being able to smell the scent proceeded with the breathing techniques.

Breathing Techniques: Three different breathing exercises using diaphragmatic breathing or “Belly Breathing” are used in the present technique. Note that those who suffer from high blood pressure, seizures, pregnant, have hernias or emotional disorders such as bipolar or ADHD may not tolerate some breathing techniques as well. The three breathing exercise are herein: 1. Nose Inhalation and progressively extended exhale through mouth, 2. Counting breathing exercise: Four (4) count nose inhalation, seven (7) count breath hold and resistant mouth exhalation (Note: Those with high blood pressure or an untreated aneurysm should not do the breath hold. In this case go directly to the resistant exhalation), and 3. Slow nostrils breathing with extended exhalation. Inhale three (3) counts, exhale (6) counts.

1. Nose Inhalation and progressively extended mouth exhale. Before entering MRI machine, once the patient is on the MRI examination table and aromatherapy administered, have patient place one hand on their stomach to ensure diaphragmatic breathing. Tell patient that their belly should rise on the inhale and fall on exhale. Begin nose inhalation and progressively extended exhalation through the mouth. Verbally instruct the patient to inhale and exhale. Tell the patient, “on your next exhalation make it longer (extend) and every exhale after this try to make is a little longer.” Continue with this breathing technique while explaining the MRI exam and preparing patient for the exam (i.e. positioning and padding of patient). After the patient is prepared ask them to breathe normally.

2. Counting Breathing exercise: This exercise is performed before entering MRI machine. Before beginning this technique ensure the patient does not have high blood pressure and/or an untreated aneurysm. Explain counting breathing exercise to patient. (Explanation) You will inhale on four (4) counts, Hold it for seven (7) and when you exhale you will place the top of your tongue on the back or your front teeth and make a “thh” sound as if you are trying to say the word “the” while exhaling. Do not stop the “thh” sound until you are out of air. Demonstrate for the patient and then ask them to copy. Inform the patient that the “thh” sound is how “I” (the technologist) will know when you are ready to inhale again. Be sure to include telling the patient not to worry about counting as the technologist will do the counting for them. Have patient place at least one hand on their belly and tell them to make sure their belly is rising on their inhale and falling on their exhale. Begin with 1 or 2 clearing breaths (inhalation followed by exhalation). Use the clearing breaths to ensure patient is using diaphragmatic breathing. (i.e. stomach rising on inhale and falling on exhale). After the last clearing breath exhalation, begin four (4) count inhalation (counting out loud and down from 4 to one). Hold (spoken) seven (7) counts (counting out loud and down from 7). Do not do breath holds on patients that have a history of aneurysms or uncontrolled high blood pressure. Now exhale making the “thh” sound (spoken). Make the sound with the patient for a couple of seconds for demonstration purposes. Complete four (4) rounds or until patient is able to do it smoothly. After breathing rounds are complete tell patient to breathe normally.

3. Slow Nostril Breathing with Extended Exhalation: Patients will be instructed to perform this breathing technique through the MRI exam. Have patient place at least one hand on their belly and tell them to make sure their belly is rising on their inhale and falling on their exhale. Instruct patient to breathe deeply, slowly and only through their nose while in the MRI machine. Instruct patient to inhale in three (3) counts and exhale in six (6) counts making it longer than their inhale. Inform the patient that the 3:6 breathing is optimal, but if it is not possible or it stressed them out, to simply make sure the exhale is longer than the inhale. Before moving patient into MRI machine inform patient that if at any time they become anxious in the MRI machine to ask one question; Am I breathing? Then instruct them to breathe, because they are probably not breathing. Remind the patient they have control over the situation and can always squeeze the emergency button to be let out of the machine at any time. Suggest that the patient keep his (or her) eyes closed during the entire exam. Communicate with the patient after each sequence and address them by name; inform them of the approximated time of next sequence along with number of remaining sequences or time.

Once the exam is completed, clear out any remaining scent by open the ceiling vent as soon as you enter the room and remove the 2×2 gauze from in front of the bore vent and dispose of it in a lidded garbage can outside of the MRI room if possible. Additionally a fragrance free odor neutralizer may be used for any lingering scent.

Physiological data is also recorded during the MRI exam at the approximate halfway point. Once the exam is completed, the patient is told to remain lying still for a minute while the post exam physiological data was recorded.

Data Analysis: There were three hypotheses tested in this study. The first hypothesis was a reduction in overall anxiety of nervous MRI would result in patients of the experimental group using aromatherapy and breathing techniques (SIGBA). The second hypothesis was, the intervention would reduce the amount of time it took the medication to take effect in patient's whose medication had not reached its maximum effectiveness before the start of their examination. The third hypothesis was that a combination of breathing techniques and aromatherapy (SIGBA) would be more effective than using aromatherapy alone.

This qualitative and quantitative data of this pilot study was analyzed separately with integration taking place in the data collection, discussion and conclusion sections of the thesis using a triangulation strategy. The qualitative data was analyzed and triangulated by the PI as well as working with statisticians to analyze the quantitative data. The qualitative portions of this study were based on the participants' answers to the open ended question on the questionnaires distributed during the study. Coding was performed using Microsoft Excel to saturate the answers into key words to further test the hypotheses (Mertens, 2005).

The Microsoft Excel database was used to analyze the qualitative data and to create visual graphs for further analysis of data and aide in its presentation. All qualitative data was transcribed into Microsoft Word 2012, and converted into a table format double and checked for accuracy before entering it into the Excel data base. Each qualitative question and sub-questions had its own worksheet in Microsoft Excel that included the following face code columns; participant ID number, group ID code, exam type, gender, age, location, medication use, medication effect, question responses and qualitative theme codes (Elliot, 2011). Each question response had its own Excel cell, but in the case of a multivalent response a separate cell was be created (Elliot, 2011). The data was then coded using a grounded analysis approach and in vivo terms and categorized according to the reoccurring themes found in the qualitative data. Once the data themes were established they were categorized and assigned a letter code for further analysis of subgroups (Bradley, Curry, & Devers, 2007; Creswell, 2009; Elliot, 2011). Once the letter codes were assigned the totals of each code was quantified for further analysis, in the form of histograms. This analysis included mapping the common themes to find relationships between the qualitative open ended questions and hypothesis using the program CompediumNG. All information was reviewed twice for accuracy, once by the PI and a second time by an independent party before analysis. This independent party was a colleague of the PI and only had access to the data without any identifiable information about the study participants.

The quantitative data was analyzed by the PI and a statistician as follows. Given the small number of participants in the study that received the SIGBA treatment n=17 and the fact that the measure was a Likert-scale assignment, it was not possible to use traditional analyses, such as t-tests and ANOVA to determine anxiety levels, as initially planned. The Likert scale means were distributed normally, that being said there were a large number of responses that were identical making traditional non-parametric tests, such as a Wilcoxon Rank Sum Test or Wilcox Signed Rank Test implausible. Therefore instead of using these tests an exact binomial distributions-based test was used to determine whether there was a reduction in anxiety and to what degree. A t-test was in fact used to test one of the hypotheses and lastly covariates and physiological data was analyzed using logical regression analysis.

The anxiety reduction measures were based on responses to the following Likert scales and were as follows. A measurement of the anxiety reduction from pre-scanning to post (SIGBA) treatment was measured. The anxiety reduction levels from post (SIGBA) treatment to average anxiety during the MRI exam were measured. Furthermore the anxiety reduction levels from pre exam to the average anxiety levels during the exam were also tested. Once these results were obtained the binary responses were measured. Lastly the control group's anxiety levels were measured in a similarly and using the same Likert scale responses.

Once the quantitative data was analyzed it was transformed into qualitative data in order to compare the qualitative and quantitative results (Creswell W. J., 2009). Furthermore the qualitative data was analyzed twice and the results of each compared for accuracy. Once accuracy of the data analysis was established triangulation of the data took place in the discussion and conclusion portion of the thesis.

There were several limitations in which the primary researcher (PI) had no control over. The first involves the study site, Rebound Orthopedics and Neurosurgery, who made it very clear that in order to obtain permission to use the facility, the PI had to be the sole person administering the interventions. The initial proposal was that the PI would administer the interventions for the experimental group while the PI's supervisor conducted the experiment on the control group and provided usual care. In essence the PI was the sole study researcher, that administered all interventions to the control and experimental groups, which was not ideal, but out of the control of the researcher.

There were several deliberate limitations on this study which included only recruiting participants who self-described themselves as claustrophobic and/or experiences anxiety during their MRI exam. An additional delimitation included recruiting participants with and without anti-anxiety medication. The reasoning for including patients who did and did not use medication was the hypothesis that breathing exercises and aromatherapy would reduce the time it took medication to take effect. Furthermore there are a limited number of studies that included patients who took medication for MRI anxiety and there was an interest in the experiences of medicated patients had during MRI exams when compared to those that did not. An additional delimitation was the principle investigator (PI) deciding to limit the study participants to one site (Portland, Oreg. Rebound site) although two sites were available through the sponsoring site. The reasoning for this decision was the significant difference between the MRI units, which may have compromised the study results and the PI only worked at the other site one day a week. The final delimitation was the use of a mixed methods methodology. This type of study design was used to help corroborate results and potentially increase the overall strength of this study. Moreover there are very few mixed methods studies on anxiety in the MRI environment and this will help contribute to gaining a more comprehensive view of the problems and solutions of the imaging modality.

Results and Discussion: The major finding of this pilot study was a 76.5% reduction in anxiety using the SIGBA technique, which validated the related hypothesis. Two other hypotheses could not be validated or rejected, but findings associated with them were beneficial.

The purpose of this study was to validate three hypotheses to determine the effectiveness of combining breathing techniques and aromatherapy (SIGBA) in reducing MRI anxiety. The first hypothesis stated that a combination of breathing techniques and aromatherapy (SIGBA) would be effective in reducing anxiety in MRI patients. The second, that SIGBA would reduce the time it took medication to take effect in patients whose medication had not reached maximum effectiveness. The third hypothesis stated that a combination of breathing techniques and aromatherapy (SIGBA) would be more effective than using aromatherapy alone. There were a total of 38 participants with n=17 in the experimental group and n=20 in the control. The experimental and control groups were divided into those with and those without anti-anxiety medication. The control group had eight (8) participants with medication and 12 without, while the experimental had five (5) with medication and 13 without.

The analysis of covariates calculated the difference in the reduction of anxiety levels based on gender, age, medication usage and previous MRI exam experience. Analysis of anxiety levels in each covariant category showed no statistically significant differences. The percentage of anxiety reduction of each covariant from pre exam to post SIGBA anxiety levels were calculated along with the difference in anxiety levels and p values. See Tables 1a.-1c. Based on gender 90% of women as opposed to 57% of men had a reduction in anxiety. Medication usage showed 80% of medicated users versus 75% of non-medicated participants had a reduction of anxiety. Analysis showed participants with prior MRI experience had an 80% reduction in anxiety as opposed to 50% of those without prior experience. Age was measured by using logical regression analysis with age as a continuous variable, resulting in no statistical differences p=0.55. In short there were no noteworthy differences in the reduction of anxiety based on age, gender, medication use, or prior MRI experience.

TABLE 1a
Pre MRI exam to Post SIGBA Reduction of Anxiety based on Gender

| Measure | Female | Male | Difference | Power |
|---|---|---|---|---|
| Proportions | 90% | 57% | 33% | P = 0.112 |
| Sample Size | 10 | 7 | | |

TABLE 1b
Pre MRI exam to Post SIGBA Reduction of
Anxiety based on Medication Use

| Measure | Medication | No Medication | Difference | Power |
|---|---|---|---|---|
| Proportions | 80% | 75% | 5% | P = 0.82 |
| Sample Size | 5 | 12 | | |

TABLE 1c
Pre MRI exam to Post SIGBA Reduction of Anxiety
based on Prior MRI Experience

| Measure | Prior MRI | No Prior MRI | Difference | Power |
|---|---|---|---|---|
| Proportions | 80% | 50% | 30% | P = 0.38 |
| Sample Size | 15 | 2 | | |

The first hypothesis that stated that combining of breathing techniques and aromatherapy (SIGBA) would reduce anxiety in MRI patients. An analysis of the differences in anxiety levels of all experimental participants' n=17 from pre examination to post SIGBA anxiety was conducted using an exact binomial test based on Liked scale responses. Responses revealed a 76.5% anxiety reduction from pre exam to post SIGBA treatment. The percentage of patients experiencing a reduction was significantly greater than 50% with p=0.02<0.05 for a 2% significance level. Likewise there was a 76.5% reduction in average anxiety during the MRI exam compared to pre exam levels. See Table 2. Furthermore based on descriptive analysis of a closed ended question that asked participants to choose which tool was most effective in reducing their anxiety, 56% indicated SIGBA as the most effective compared to 39% for breathing exercises and 6% for aromatherapy.

TABLE 2
Percentage anxiety Reduction¹: Column 1; Pre-SIGBA/MRI to Post SIGBA Treatment, and; from
SIGMA Treatment to During MRI scan, Column 2; Comparison between Experimental (SIGBA)
Treatment to Control for Percent Anxiety Reduction from Pre-SIGBA/MRI to During MRI Scan

| | | | 2. Experimental VS Control Pre SIGBA | |
|---|---|---|---|---|
| | 1. Experimental Group Anxiety | | to During MRI Anxiety Reduction | |
| | Reduction | | | Control During |
| | Pre SIGBA/Exam (a) | Post SIGBA to | Experimental Pre SIGBA to | MRI Anxiety |
| Measure | to Post SIGBA (b) | During MRI (b) | During MRI (a) | Reduction (b) |
| Percentage Experiencing Reduction | 76.5%** | 47.3%** | 76.5%*** | 66.7%*** |
| Estimated Significance Level (p)* | 0.02 | 0.69 | 0.02 | 0.12 |
| (a) First Mean Likert Measure | 4.88 | 3.21 | 4.88 | 4.78 |
| (b) Second Mean Likert Measure | 3.21 | 2.88 | 2.88 | 3.33 |
| (a) − (b) Reduction to Likert Scale Measures | 1.68 | 0.32 | 2.00 | 1.44 |
| Number of Subjects | 17 | 17 | 17 | 18 |

*Based on exact binomial test
**Treated subjects measured for both Pre Exam to Post SIGBA. Comparison and Post SIGBA to During MRI Comparison
***Experimental and Control patients both measured for Pre Exam to During MRI. Even though treated group's percentage is significantly greater than 50% the Control is not, the two estimates do not significantly differ from each other (p ≈ 0.51)
¹Note: In addition to the percentage of patients experiencing reductions are given along with estimated probabilities of Type 1 Errors, the actual mean Likert estimates and their differences are given even though statistical tests were not made.

These findings were corroborated with qualitative data where 33% of the experimental (SIGBA) group said their anxiety was lessened, compared to 22% of the control group. Participants were asked to respond to the open ended statement “In your own words please describe your overall MRI experience today regarding anxiety” The most prevalent themes amongst the experimental groups were that they experienced “very little anxiety” amongst the group with medication and “nervous before/Ok during” for the non-medicated group.

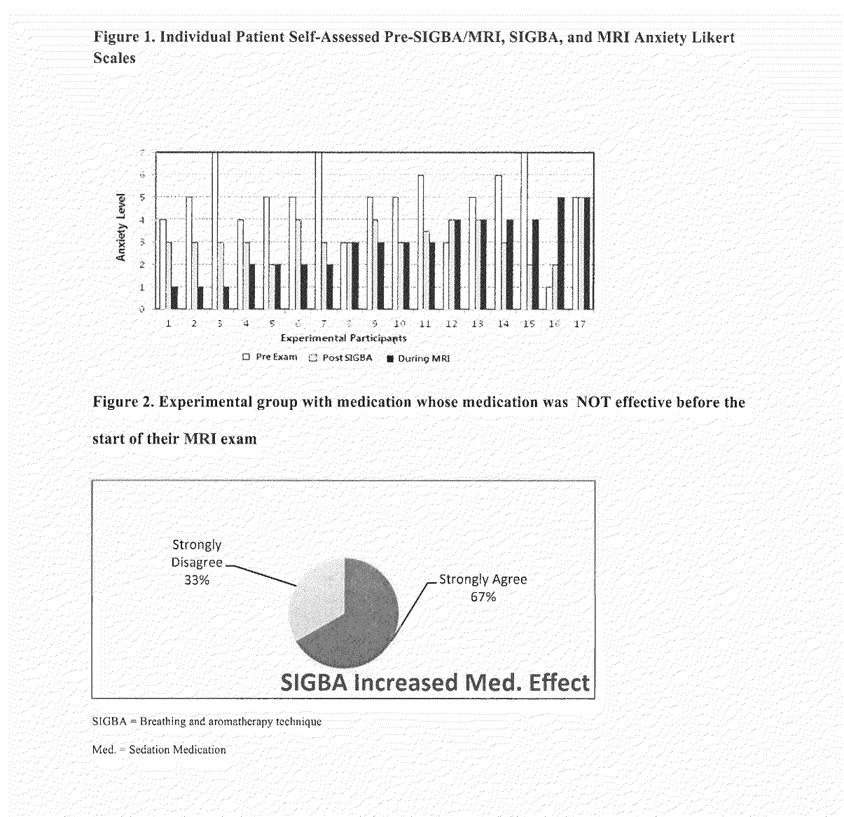

Furthermore the post SIGBA anxiety levels were measured against the average anxiety levels during the examination. Results showed an additional 47% reduction in anxiety, which was not statistically significant with a p=0.69>0.05. (See Table 2) That being said, based on the Likert scale answers there was an actual estimated average reduction in the Likert Scale for both columns, a large average reduction of nearly 1.7 for the SIGBA group and a small reduction estimated at just over 0.32 for the average anxiety while in the MRI unit. To better demonstrate the relationship between the pre and post SIGBA subjects and MRI average anxiety during exam, the Likert scale responses are displayed in FIG. 1.

A comparison of anxiety levels between the experimental (SIGBA) and control group was also completed. This comparison was of the percentage reduction in average anxiety during the MRI exam between SIGBA-treated and control patients based on exact binomial testing. The SIGBA participants showed a 76.5% reduction in anxiety while the control experienced a 66.7% reduction. The difference in anxiety reduction of the SIGBA group was not statically significant from the control with p=0.51>>0.05, but the power of the reduction seen in the control group was p=0.12, which is not a significant finding. See Table 2 above.

Physiological data was compared to the average anxiety levels of the SIGBA treated group n=17 using regression analysis. Although there were two physiological measures taken, breathing and heart rate. The physiological measures used for analysis was the heart rate, as the experimental group did breathing exercise throughout the entire exam. These results showed a significant decrease in the percentage of anxiety reduction of two measurements. An increase in the physiological measure for both the Pre-SIGBA to SIGBA treatment and the SIGBA treatment to during MRI were assessed. Negative logistic linear coefficients indicated an increase in the physiological measure were associated with a decrease in the percentage of reduction in anxiety. The physiological measurements of Pre-SIGBA to SIGBA treatment and SIGBA treatment to during MRI anxiety level reduction was significant with a p=0.02 for each respectively. In essence there was an inverse relationship, as the physiological measures went up; the average level of anxiety reduction went down (see FIG. 1).

Based on the statistical analysis as described above there was a statistically significant reduction in anxiety of MRI patients who received the SIGBA treatment. Although there was no statistical significant findings found between the control and experimental groups (SIGBA), there was a reduction of approximately 10% and to a greater extent in the SIGBA treatment patients thus was concluded that the hypothesis that MRI patients would have a reduction is can be validated.

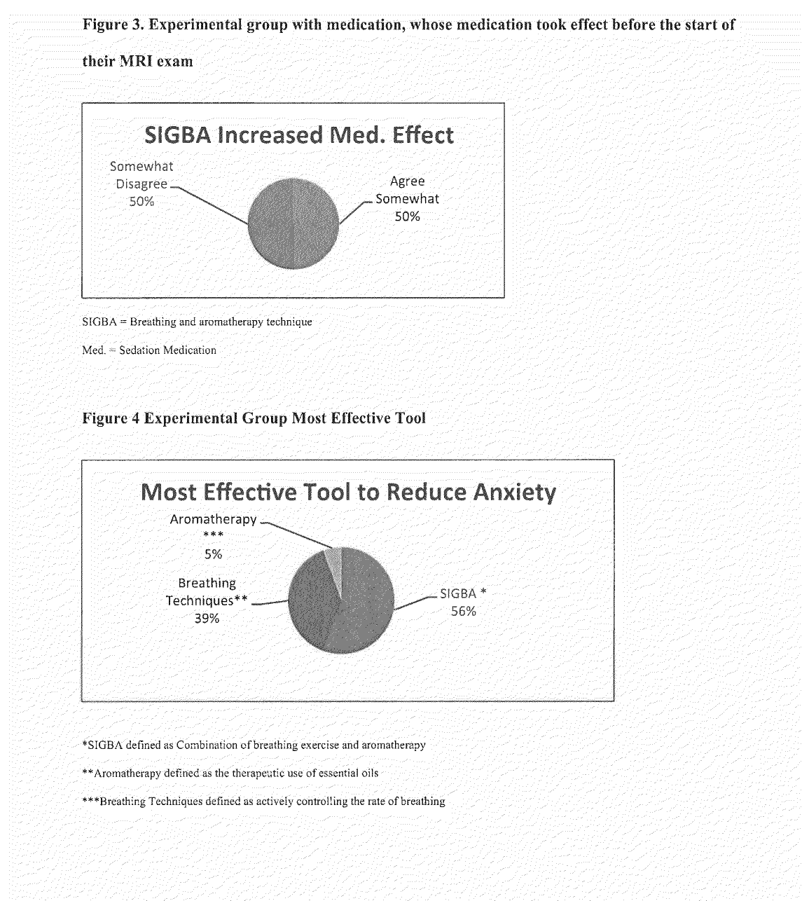

The second hypothesis evaluated whether SIGBA helped reduce the time medication took effect in patients whose medication had not reached maximum effectiveness. This hypothesis could not be accurately tested due insufficient data. That being said, there was data available on how patients felt SIGBA helped increase the medication effectiveness. This was tested using the answers of two questions. The first was an account of MRI patients whose medication had not taken effect, deemed by the responses to the question “Do you feel the medication has taken affect?” with the choices of Yes, No and Somewhat. All answers other than yes were considered as the medication had not fully taken effect. All no responses of experimental and control groups were compared to with their average anxiety level during their MRI exam using a t-Test two sample assuming unequal variances. Participants whose medication had not taken effect in experimental group n=4, and control whose medication had not taken effect n=5 with the exclusion of one participant due to non-response. The average level of anxiety mean was of 3.25 and 3 and standard deviation of 1.3 and 1.5 respectively, resulting in a p=0.8, showing no statistically significant difference between these two groups. Descriptive analysis confirms this as a histogram showed 75% of the experimental group whose medication had not taken effect having some form of anxiety. The control group had 60% with some form of anxiety. Conversely descriptive data showed 67% of the medicated experimental whose medication had not taken effect group n=3. Two (2) responded that they strongly agreed with the Likert scale statement, “The aromatherapy and breathing exercises (SIGBA) increased the relaxation effect of my medication.” compared to one (1) who strongly disagreed (FIG. 2.). Additionally while those whose meds did take effect n=2 with one (1) who agreed and the other disagreed (FIG. 3).

In reference to qualitative data, none supporting or disproving the validity of this hypothesis were observed. Thus in conclusion, results suggested SIGBA may have increased the relaxation effect of medication. That being said, and based on the lack of either qualitative or quantitative data to test if SIGBA decreased the effective time of medication the analysis this hypothesis cannot be rejected or accepted.

The third and final hypothesis evaluated whether a combination of breathing techniques and aromatherapy (SIGBA), was more effective than using aromatherapy alone. There was not sufficient qualitative data available to completely evaluate this hypothesis, although there was some information available for partial analysis. Moreover there was no experimental group in which aromatherapy was the sole intervention, thus this study provided no data in which the SIGBA treated group could be compared against and testing the hypothesis was not possible.

Although hypothesis testing was not possible there was descriptive qualitative data related to the hypothesis. One closed ended question posed to both experimental groups with and without medication asked, what they felt was most effective in reducing their anxiety. The choices were; aromatherapy, breathing exercises, both equally and neither. Data was analyzed using histogram in measuring the frequency of each response. Sixty seven percent (67%) of the experimental without medication n=12 responded that the breathing and aromatherapy (SIGBA), for was the most effective tool, followed by breathing exercises at 25% and aromatherapy alone at 8%. The experimental group with medication n=5 felt the breathing exercise alone was the most effective tool followed by SIGBA at 20% and 0% for aromatherapy. Cumulatively experimental participants n=18 results where 56% felt SIGBA was the most effective tool in reducing their anxiety followed by breathing exercise at 39% and lastly aromatherapy at 5% (FIG. 4).

In conclusion, and based on descriptive analysis that suggested SIGBA was a more effective tool in reducing anxiety compared 5 to aromatherapy alone. That being said and based on the unavailability of substantial qualitative data or comparison groups to definitively test this hypothesis; this hypothesis can be neither accepted nor rejected.

An analysis of the control group's intervention results were as follows. The control group was provided with, an information sheet as their main intervention explaining the MRI procedure, along with sham aromatherapy. There was no significant decrease in average anxiety for the control group, either with or without mediation. It must be noted that a significant difference may not have been detected due to small sample size. While most, 76% felt this information helpful in exam preparation, 47% felt the information sheet it, along with the sham aromatherapy was not useful in reducing their anxiety. Thus it can be concluded that SIGBA was more effective than the information sheet at reducing anxiety. These results were consistent with other studies that used written information as an intervention (Bolejko et al., 2008; Törnqvist et al., 20 2006).

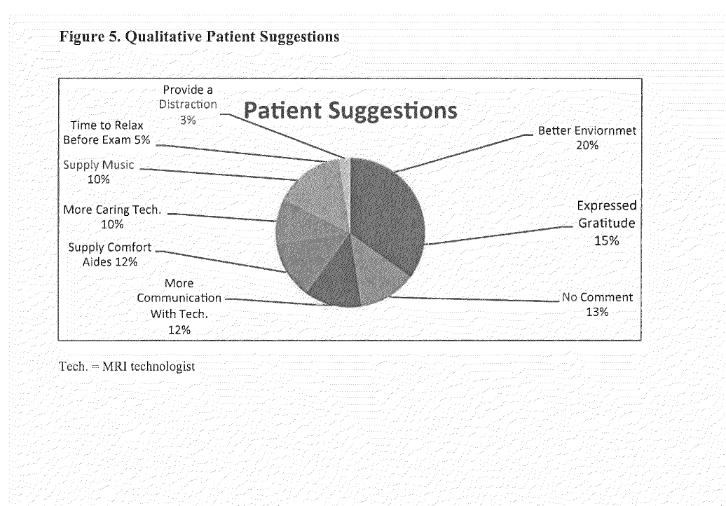

Lastly there was a finding unrelated to the hypothesis that is noteworthy. A qualitative statement in which patient were asked to give suggestions on making MRI exams more comfortable was included as an open ended question. The purpose of including this question was to give patients a voice as they are the best source for shedding light on making the MRI experience better. Notice that the top five suggestions excluding the no comments and expressed gratitude were: 1. providing a better environment including having “more light” and “cool free flowing air”, 2. more communication with the technologist, 3. supply comfort aides, such as a “warm blanket” or “eye pillow”, 4. a more caring technologist, and 5. supplying music (FIG. 5).

In the first clinical study in which a combination of breathing exercises and aromatherapy (SIGBA) was been used to reduce anxiety in MRI patients revealed a reduction of 76.5% in patients. This statistically significant reduction, p=0.02 in anxiety was seen both before entering and while in the MRI unit. There may have been several factors that contributed to this result. The first may have been combining both breathing and aromatherapy as a single intervention. The approach of using more than one interventions was suggested in the first study to test scent for reducing MRI anxiety conducted by Redd et al 1994. This study's authors suggested an additional cognitive coping mechanism could further reduce anxiety in patients. This same study also suggested a choice in scent would also contribute to reducing anxiety further (Redd et al., 1994). This current study used both of these suggestions which resulted in 76.5% average reduction during the exam compare to 63% in the Redd (1994) study.

Moreover both breathing exercises and aromatherapy are considered cognitive based CAM modalities, with the ability to change a person's thoughts and served as distraction techniques. Qualitative findings supported this as cognitive changes in regards to aromatherapy and the breathing exercises were recurring themes. In regards to aromatherapy, patient statements included “Very helpful in getting me to relax.”, “Felt more like I was at a spa than an MRI” and “Made me think of pleasant things and not doing an MRI. It helped me relax”. Patient's had this to say about breathing techniques; “Breathing exercises helped provide focus during exam . . . helped distract, lowered anxiety” and “Focusing on breathing kept my mind from racing. Helped with the panic”. Lastly both CAM modalities have been shown to positively affect mood and elicit physiological changes to support anxiety reduction (Arch & Craske, 2006; Brown & Gerbarg, 2005a; Hongratanaworakit, 2004; Warrenburg, 2005). This may be the reason physiological data supported a reduction of anxiety.

The implications of these results for MRI imaging are significant as anxiety is a major issue for patients, technologists and imaging departments. Imaging departments experience increases in operational costs from cancelled MRI scans due to anxiety (Bell, 1996). Although the 10% anxiety reduction difference seen between the experimental and control groups was not statistically significant, such a decrease could equate to great saving in operational costs when considering real world applications. Likewise, with the changing face of healthcare becoming more socialized and a significant reduction in Medicare reimbursements having took place effect earlier this year, the importance of controlling anxiety in the MRI environment is essential (Watson, 2014).

Implications for the patients are clear in regards to their healthcare. A more relaxed patient during their exam will enable them complete the test the first time, allowing then to heal sooner due to a faster diagnosis. Moreover the quality of care also increases when a medical procedure causes less psychological distress and may also increase customer satisfaction.

The hypothesis that assessed whether SIGBA would help reduce the time medication took effect anxiety in patients whose medication had not reached maximum effectiveness, could not be rejected or accepted. There are several reasons for these results. The primary reason was a lack of data available to test the hypothesis. Accurately testing this hypothesis would have required more advanced instrumentation than what was available for this pilot study. The secondary was the sample size of the medication group without an effect was too small to accurately test.

While the data provided could not prove this hypothesis correct, available data suggested SIGBA may have increased the effect of the participant's medication. The experimental group whose medication had not taken effect before the start of their exam, and to a lesser extent for those whose medication had taken effect stated SIGBA help increased its effect. This also suggested the aromatherapy may act as a complement to medication. As stated above this was based on a small sample size and more research with a larger sample size and comparison group would be necessary to definitively prove this effect.

The third and final hypothesis discussed assessed whether a combination of breathing techniques and aromatherapy (SIGBA) was more effective than using aromatherapy alone. This hypothesis could not be accepted or rejected based on the current study data, as there was no intervention group in which aromatherapy was the only intervention. Although this hypothesis was not proven or disproven, there was data that suggested SIGBA may have been more effective than aromatherapy alone based on participant's responses, but in order to conclusively make this claim additional data and hypothesis testing in necessary.

With the absence of an experimental group who received only aromatherapy to compare with the SIGBA treated, the only comparisons available would be that of another studies, where scent was the only intervention. One such study meeting this criterion was conducted by Redd et al 1994, in which a synthetic vanilla scent was used. Although comparisons of results between these two studies are anecdotal due to inherent differences, and cannot conclusively say whether or not the use of SIGBA is more effective than using a scent alone. Comparing them adds value to the current study by providing comparative results.

Looking first at the similarities of these studies, the number of experimental participants are relatively close, with the current study n=18 and Redd study n=20. Both studies used VAS as a way of measuring the level of anxiety before and during the MRI exam, the Redd study used a 100-mm VAS and current study a 7 point Likert scales (Redd et al., 1994). These two scales have been found to have no statistically significant differences in responsiveness, thus comparison can be comfortably made (Jaeschke, Singer, & Guyatt, 1990). The biggest differences in these studies the difference in anxiety levels were related to the of the aroma's pleasantness before and after MRI exam, whereas the current study used the difference between pre-exam and post (while still on MRI table) SIGBA anxiety levels without regard to scent pleasantness. Comparing the administration of scent, the Redd study used the researchers chosen scent, a synthetic vanilla scent administered via nasal cannula with humidified air for intervention and the control who received only humidified air (Redd et al., 1994). The current study used 100% pure essential oils administered using a 2×2 gauze with participants chosen scents that was placed in from of the air vents of the MRI machine with for the experimental and the control group who received sham (grape seed oil) aromatherapy. The Redd study patients experienced an average of 63% less anxiety, while the current study participants experience an average of 76.5% less anxiety.

Although there was no data available to either support or reject this hypothesis, based on comparing the results of this pilot study to a similar study, using a combination of aromatherapy and breathing techniques (SIGBA) may be more effective than using aromatherapy alone. To definitively confirm this suggestion, future studies to include a SIGBA and aromatherapy only group for comparisons is necessary.

Conclusions: The results validated the first hypothesis that stated combining breathing exercise and aromatherapy (SIGBA) would reduce anxiety in MRI patients on a statistically significant level. The other two hypotheses could not be validated due to insufficient data. One hypothesis stated that combining breathing techniques and aromatherapy would be more effective than using aromatherapy alone. Although this hypothesis could not be validated, it was viewed by participants as the most effective tool in reducing anxiety compared to aromatherapy and breathing techniques alone. Furthermore there was a significant reduction in anxiety using SIGBA when compared to another study that used scent alone as an intervention. The other stated SIGBA would decrease the time it took for sedation to become effective in patients whose medication had not reached its maximum effectiveness before their examination time. While this hypothesis was not validated, patients strongly agreed that SIGBA helped increased their effect medication.

There were three major results found in this study in relation to the hypothesis. The first was SIGBA was found to be overwhelmingly effective in reducing anxiety in the experimental group. When comparing the difference of anxiety levels between the experimental and control groups the results trended towards being significantly different but fell a bit short. This result was not expected and upon reflection of qualitative data the control groups seemed more dependent on their interaction with the technologist. The two common qualitative themes amongst this group with and without medication were that the technologist was a major factor in helping reducing their anxiety. This observation was strengthened as there were no obvious variables in the care they received, as the control groups had the same technologist during their exams. Another noteworthy observation was the control group received sham aromatherapy. As expected the majority said they did not smell any scents, yet there were a few who claimed it was very helpful, which suggests a placebo effect may have played a role in the insignificant difference in anxiety between the experimental and control groups.

The second major finding was that although there was not sufficient data to formally test whether SIGBA or aromatherapy alone was more effective. Available data suggested it was more effective in reducing anxiety then aromatherapy alone. The majority of participants 56% noted the SIGBA treatment as being the most effective tool that helped reduce their anxiety and qualitative data showed the most common themes were that the aromatherapy was relaxing and breathing techniques helped decrease anxiety. In short these finding were promising, yet not definitive. In order to conclusively say SIGBA is more effective than aromatherapy alone further studies are necessary comparing these two interventions.

Although the SIGBA technique could not be validated in decreasing the time it took medication to take effect, patients whose medication had not taken full effect strongly felt it helped increase the effect of their medication. There were two interesting findings associated with the use of medication and the SIGBA technique. Firstly whether or not SIGBA can increase the effect of medication could not be corroborated and remained elusive, findings does suggested the SIGBA technique may complement the use of medication to reduce MRI anxiety.

The second dealt with the general use of medication to reduce anxiety. Sixty seven percent of medication users said their medication had not taken full effect before the exam began. The average time medication was taken before the MRI exam was 45 minutes. With well over half of patients whose medication was non-effectiveness before the start of their exam. This poses a question as to how long before the start of their MRI exam should patients take their medication. This is a very important question because if the medication has not taken effect before the exam begins then prescribing it is a moot point. Although more research on this topic is necessary findings in this study suggested patients should take their medication more than 45 minutes before the start of their exam for maximum effectiveness.

Lastly the suggestions from study participants on how to make the MRI experience better were informative and these suggestions should be taken seriously as the patients are the most knowledgeable of their needs in the MRI environment.

There were two theoretical foundations of this study and the results supported both. The first theoretical foundation was the integrative medicine theory, based on the belief that treating patients with an integrative style creates a more holistic approach to medicine (Miller et al., 2011). This current study used this holistic approach two-fold. The first was combining two complementary alternative medicine (CAM) modalities, aromatherapy and breathing techniques and the second, was joining the conventional orthodox medicine (COM) approach to MRI anxiety, i.e. prescribing medication and combining it with the CAM modality breathing techniques and aromatherapy (SIGBA). The significant reduction in anxiety using the combined CAM modalities, along with the fact that the majority of experimental participants felt the combination of aromatherapy and breathing techniques were the most effective tool in reducing their anxiety, compared to aromatherapy or breathing techniques alone helped supports the integrative theory. Likewise in the case of combining CAM modalities and COM approaches, the majority of participants who took medication in the experimental group strongly agreed the SIGBA technique helped increase the effect of their medication. In short incorporating both COM and CAM approaches to MRI anxiety showed significant results and support the integrative theory approach to medicine is superior to either one alone.

The second theory involved integration of the humanistic and cognitive theories as an approach to help reduce MRI anxiety was supported by this research. This theory was based on an approach to psychological counseling and grounded on establishing a positive and safe relationship with the patient to help change their negative cognitions (Miller et al., 2011). The integration as applied to this research was the idea that if the technologist established good rapport with the MRI patient in combination with the breathing and aromatherapy (SIGBA), it would be effective way of dealing with their anxiety. This theory was applied to MRI anxiety, because past research showed cognitions and the technologist/patient relationship significantly influences the patient's nervousness (Thorpe et al., 2008). The qualitative research showed reoccurring themes that supported this theory. Both the experimental and control groups felt the technologist helped ease their anxiety, (humanistic theory) while common themes in reference to aromatherapy and breathing techniques was they helped change the patient's focus away from their fears (cognitive theory). Thus this theory albeit in a different realm of counseling was supported by the results of this research.

The result of this study is in congruence with other studies that explored interventions to reduce anxiety in the MRI environment. The most comparable research to the current was conducted by Redd et al 1995 in evaluating the effect scent had on MRI anxiety. Similar to this study a significant reduction in the average anxiety was seen in the experimental group. This study found a 63% reduction while the current study revealed a 76.5% decrease (Redd et al., 1994). There were two significant differences in the Redd study compared to the current research. The first was the current research intervention involved combining breathing exercises with the aromatherapy, while the Redd study only used aromatherapy. Likewise there was a singular synthetic vanilla scent used compared to the current study that used blended pure essential oils, in which patients could choose from. Ultimately both of these studies showed there were benefits to using scent in the MRI environment.

A more current study to additionally compare the results of this study was conducted by Schellhammer et al 2012 in which scent was examined as an intervention to reduce motion artifacts from psychological stress. Unlike the present study the Schellhammer study did not find any significant findings to suggest scent was helpful in reducing stress and increasing patient's wellbeing during a MRI procedure (Schellhammer et al., 2013). The scents used in this study were said to be “aromatherapy”, but in actuality in order to be truly considered aromatherapy essential oils must be used (Price & Price, 2007). The scents used in this study were commercial scents used to fragrance tanning beds and elevators and may have been the reason this study was not able to duplicate the anxiety reducing effect seen in the present study. Additionally this study only used scent as an intervention instead of incorporating additional relaxation techniques as suggested in the Redd (1994) study.

Finally a pilot study conducted by Argue 1995 that examined the effects of physiological interventions on MRI anxiety was compared to the present study. This study was similar to the current research as both used interventions aimed at changing destructive cognitions that caused anxiety and exam cancellations. The results of the Argue (1995) study showed a more significant reduction in MRI anxiety using a combination of cognitive interventions when compared to just using one (Argue, 1995). Findings of this study were similar to the current research as both suggested combining the cognitive interventions, aromatherapy and breathing exercises (SIGBA) was more effective than either one alone. That being said, a noteworthy difference in these studies is the Argue study, unlike the current study did not purposefully use patients known to have anxiety and felt caution should be taken with the results as the target audience (claustrophobic patients) may not have been well represented (Argue, 1995).

This current study has broad implications on a global perspective relevant to the industry standards of how anxiety in MRI medical imaging is dealt with, and how technologists are trained to deal with this issue. Anxiety in MRI departments is a global issue that is apparent from the multitude of studies conducted on this subject from all over the world. There were studies from Malaysia, Germany, to Australia and many places in between (Munn & Jordan, 2013; Sarji et al., 1998; Schellhammer et al., 2013). Based on the significant effectiveness of the SIGBA treatment as well as the low cost of implementation the potential for this treatment to spread on a global scale is conceivable.

The production of MRI machines are global with manufacturers not just in The United States of America, but also includes international companies such as Phillips from Netherlands, Siemens, based out of Germany and the Japanese company Hitachi. These companies have recognized MRI anxiety as an issue and have taken on the role of creating high technological solutions such as installing viewing screens, and virtual reality technology into their MRI systems. While these high tech solutions have value the SIGBA technique contributes to increasing the value of low tech, skill based solutions. Such low tech solutions can be coupled with high tech solutions in creating a more holistic approach to MRI anxiety. For example manufactures could create an optional feature to their systems that aide in administering aromatherapy effectively and maximizing its therapeutic value. Likewise an MRI anxiety reduction sound option packet could be created with pre-recorded breathing techniques available to patients via the sound system. Such recordings are currently available as a standard feature on MRI machines to reduce breathing motion on specific exams such as lung and abdomen scans, thus creating one especially for anxiety reduction is probable. In short there are a number of global implications this study may have on the MRI manufacturing industry on a global scale. Small changes such as those discussed above could potentially have a significant impact on the number of cancelled MRI exams seen all around the world.

Lastly, the versatility of possible scents used in the SIGBA technique can be adapted to fit any ethnic group or nationality, broadening its global reach because its adaptability is the key to this technique having a global impact. Different cultures have diverse associations with particular scents. For example lavender is associated with death in certain countries and may not be effective in reducing anxiety, whereas in others the associations are more positive (Lawless, 1994). Furthermore this technique is skill based and inexpensive to implement potentially broadening its global reach to less developed countries. In summary, the possibility of SIGBA playing a role in reducing anxieties in MRI departments around the world and having a global effect is possible.

The relevance of the social context of this study is clear in that it helps reduces anxiety in claustrophobic MRI patients and thus improving the quality of healthcare in diagnostic imaging. This improvement in healthcare can be seen in a variety of ways. This includes providing an alternative to taking narcotics for MRI anxiety, and patients who cannot take sedatives due to allergies and sobriety issues. Another involves the cost saving of decreasing cancelled MRI exams its impact on healthcare costs in The United States of America. With the trend towards socialized healthcare, the availability of advanced imaging such MRI may become limited due to long waits before patients can have these exams, as seen in other countries with social medicine such as Canada (Emery et al., 2009) Hence increasing the completion rate of this exam may have a positive effect on increasing efficiency and help control national healthcare costs in addition to increasing the availability of this imaging modality more patients. In summary the social context of this study involves increasing the quality of healthcare to MRI patients and may help control healthcare costs by reducing cancelled MRI exam due to anxiety.

This study is relevant as it contributes to the literature on MRI anxiety and provides an additionally needed intervention to reduce canceled MRI exams (Tischler et al., 2008). Additionally it has the potential to contribute to increasing the skill set of MRI technologists, thus increasing their profession's legitimacy. Lastly by providing an additional choice to help patients relax and complete their exam it provides a platform for self-efficacy and thus self-regulation without the use of drugs.

This research contributes the MRI imaging profession in several ways. To begin this research contributes to past research that recognized the role cognitions play into MRI anxiety and provides an evidence-based solution. Based on research MRI technologists often encounter patients who suffer from psychological distress on a regular basis making it evident that skills in calming patients fears would be useful (Argue, 1995; Thorpe et al., 2008). SIGBA as an intervention addresses some of the psychological needs of patients and has the potential to increase the quality of patient care. The current research helps the profession by reinforcing the importance of the relationship between the technologist, and patient as being an integral part of the MRI experience. This helps the profession as more technologists become aware of their role they play in helping control anxiety there may be an increased focus on interpersonal relationships.

MRI anxiety is dealt with differently around the world and creating more standardized solutions such as the SIGBA technique enables a benchmark to be established. By creating a basic standard, technologist can help reduce patient anxieties during their exam and increases the technologist's skill level therefore bringing the profession to a higher level of authority. Based on personal experience this is necessary, as the lay person and even other medical professionals do not have a true understanding of the MRI technologist's job and sometimes viewed us simply as button pushers. By establishing a benchmark the MRI profession may be viewed more skillfully on a global level.

Incorporating the skills required to effectively deal with the psychological component of MRI anxiety may help elevate this profession on a global scale. A psychological component of MRI anxiety exists and the most logical step is incorporating skills in MRI technologist training programs that officially address this issue (Argue, 1995; R. Lukins et al., 1997). As mentioned earlier a connection between a person's cognitions has been established in the current study results along with others (Miller et al., 2011). Thus more technologists training that focus on effectively changing anxious thoughts should take priority. In short this research may help create opportunities for technologists to expand their knowledge base, which may potentially bring the profession to a higher level of proficiency and respect.

The best way of implementing the results of this research into practice is through education. Based on the idea of creating a benchmark the best way of implementing the SIGBA technique is to incorporate it formal MRI training programs and continuing education courses for already practicing technologists. As described in prior research more interventions aimed at reducing MRI anxiety and sedation use are necessary (Munn & Jordan, 2013; Tischler et al., 2008). Implementing the results of this research in formal MRI training programs can be accomplished by the creating an MRI patient care module, which was a major goal of conducting this research. From personal experience the main focus of MRI programs is learning the technical and scientific components of the profession. Very little has been taught about the specific physical and psychological patient care needs in the MRI environment. Patient care in this environment is just as important as the science and incorporating it into training programs is an avenue of equalizing the significance each one plays in the MRI profession.

A continuing education course for already practicing MRI technologist is another way the lessons and information from this research can be implemented. All registered imaging technologists are required to obtain continuing education credits in order to maintain their credentials. Thus using that as a platform to implement the ideas found in this research is obvious and enables exposure to a larger audience. Application can also take the form of conducting in house continuing education programs or self-study programs available to individual technologists. Both of these venues are viable options as continuing education training to improve efficiency is a normal practice in many medical facilities and have been found to improve patient care and professional practices (Forsetlund et al., 2009). Facility provided continuing education courses are not always offered, and in this case self-study programs could be invaluable.

Lastly implementation of the SIGBA technique is relatively inexpensive and can potentially lessen financial losses from cancelled MRI exams (Bell, 1996). The high effectiveness of this technique may help drastically reduce cancelled MRI exam due to anxiety. The savings in operational costs from having an increased exam completion rate benefits the facility and patients alike. Furthermore implementation of this technique in the MRI is less expensive initially and in the long term due to it being more skill than technologically based. The initial investment would be purchasing the aromatherapy oils and training the technologists. Once the skill is learned maintaining it through practice and retention of trained technologists is all that is required. When comparing this to the costs of maintaining an entertainment system within an MRI unit, there can be considerable savings in investing in a skill rather than a machine.

The terms and expressions which have been employed in the foregoing specification are used therein as terms of description and not of limitation, and there is no intention in the use of such terms and expressions of excluding equivalents of the features shown and described or portions thereof, it being recognized that the scope of the invention is defined and limited only by the claims which follow.

What is claimed is:

1. A method for reducing patient anxiety during MRI treatment, the MRI treatment including a MRI scanning table and breathing bellows, the method comprising:
providing at least one aromatherapy scent;
applying the at least one aromatherapy scent in front of an air vent located on the MRI machine;
and instructing the patient to use at least one breathing technique.

2. The method of claim 1 wherein the applying further comprises placing at least one drop of the aromatherapy scent on a gauze and placing the gauze and scent in front of an air vent located on the MRI machine;

3. The method of claim 2 wherein the placing further comprises placing the at least one drop of the aromatherapy scent on a 2×2 inch gauze.

4. The method of claim 1 wherein the aromatherapy scent is selected by the patient.

5. The method of claim 1 wherein the breathing technique further comprises instructing the patient to use at least one breathing technique comprising belly breathing by inhaling through the patient's nose.

6. The method of claim 5 wherein the breathing technique further comprises instructing the patient to use at least one breathing technique comprising progressively slowing exhalation through the patient's mouth.

7. The method of claim 1 wherein the breathing technique further comprises instructing the patient to perform a counting breathing exercise comprising a four-count nose inhalation and a seven-count mouth exhalation.

8. The method of claim 1 wherein the breathing technique further comprises instructing the patient to perform a breathing exercise comprising slow nostril-breathing by inhaling for three counts and exhaling for six counts.

9. The method of claim 1 further comprising;
instructing the patient to begin a breathing protocol before entering the MRI machine; ensuring that the patient is using diaphragmatic breathing.

10. The method of claim 1 further comprising:
performing the breathing technique during the MRI treatment.

11. The method of claim 2 further comprising:
clearing out any remaining scent and removing the 2×2 gauze from the MRI machine.

12. The method of claim 1 wherein the breathing technique further comprises:
instructing the patient to use at least one breathing technique comprising belly breathing by inhaling through the patient's nose and progressively extending and slowing exhalation through the patient's mouth;
instructing the patient to perform a counting breathing exercise comprising a four-count nose inhalation and a seven-count mouth exhalation; and
instructing the patient to perform a breathing exercise comprising slow nostril-breathing by inhaling for three counts and exhaling for six counts.

13. An apparatus for reducing patient stress in an MRI environment, comprising
an MRI scanning table, the table having an air vent,
a gauze attached to the air vent, the gauze capable of accepting aromatherapy scent, and
at least one aromatherapy scent.

14. The apparatus of claim 13 wherein the at least one aromatherapy scent includes a blend of aromatherapy scents.

15. An apparatus for reducing patient stress in an MRI environment, comprising
an instructional recording of breathing techniques playable on an MRI sound system.
